(19) United States
(12) Patent Application Publication
(10) Pub. No.: US 2026/0357227 A1
(43) Pub. Date: Nov. 12, 2026

(54) INSULIN SENSITIZERS FOR REVERSAL OF INSULIN RESISTANCE

(71) Applicant: The Regents of the University of California, Oakland, CA (US)

(72) Inventors: Michael Karin, La Jolla, CA (US); Li Gu, San Diego, CA (US); Yahui Zhu, San Diego, CA (US)

(21) Appl. No.: 19/102,804

(22) PCT Filed: Aug. 11, 2023
(86) PCT No.: PCT/US2023/030076

§ 371 Date: Feb. 10, 2025

Related U.S. Application Data
(60) Provisional application No. 63/397,615, filed on Aug. 12, 2022.

(51) Int. Cl. C12N 9/16; A61K 38/00; C07K 14/005

(52) U.S. Cl. CPC C12N 9/16; C07K 14/005; C12Y 301/03011; A61K 38/00; C07K 2319/00; C12N 2740/16022

(57) ABSTRACT

This disclosure provides an isolated recombinant peptide comprising one or more of the FBP1 E1-E7 fragment or an equivalent thereof and a cell penetrating peptide, compositions and methods of using same to reverse insulin resistance in a subject in need thereof.

### CROSS-REFERENCE TO RELATED APPLICATION

This application claims the benefit under 35 U.S.C. § 119 (e) of U.S. Provisional Application Ser. No. 63/397,615, filed Aug. 12, 2022, which is incorporated by reference in its entirety.

### STATEMENT OF GOVERNMENT SUPPORT

This invention was made with government support under DK120714, CA234128, DK133448 awarded by the National Institutes of Health (NIH). The government has certain rights in the invention.

### BACKGROUND

Insulin is the master regulator of glucose homeostasis, keeping its blood concentration within a narrow range during fasting and feeding. A major site of insulin action is the liver, where it stimulates glucose uptake, glycolysis, and glucose conversion to fatty acids (FA) and glycogen, while inhibiting glucose output by blocking glycogenolysis (GL) and gluconeogenesis (GNG). How these activities are integrated and regulated to prevent fatal hypoglycemia and avoid hepatosteatosis and hyperlipidemia, metabolic disorders that affect 30% of American adults, is not entirely clear. The key insulin effector that controls glucose and lipid metabolism during the fed state is the AKT-phosphoinositide-3-kinase (PI3K) pathway, but whether and how AKT is specifically deactivated during fasting is unknown. Thus, a need exists in the art to treat diseases and/or conditions relevant to the aforementioned activities, and this disclosure satisfies this unmet need.

### SUMMARY OF THE DISCLOSURE

In one aspect, this disclosure provides an isolated recombinant peptide comprising, or consisting essentially of, or yet further consisting of, one or more of the FBP1 E1-E7 fragment or an equivalent of each thereof and a cell penetrating peptide. Also provided herein are polynucleotides encoding such recombinant peptides.

In a further aspect, this disclosure provides a vector comprising, or consisting essentially of, or yet further consisting of, the polynucleotide as described herein. In a further aspect, this disclosure provides an isolated host cell comprising the recombinant peptide described herein, the polynucleotide described herein, and/or the vector described herein. Also provided herein are compositions comprising the recombinant peptide described herein, the polynucleotide described herein, the host cell as described herein and/or the vector described herein.

In another aspect, this disclosure provides a method to disrupt the interaction of AKT and/or PP2A-C and disrupt their interactions with FBP1, the method comprising, or consisting essentially of, or yet further consisting of, contacting a composition comprising AKT and/or PP2A, and FBP1 with an effective amount of a recombinant peptide described herein or an FBP1 E7 fragment or an equivalent of each thereof.

In a further aspect, this disclosure provides a method for one or more of: activating AKT, sensitizing insulin, or reversing obesity-induced glucose intolerance in a subject in need thereof, the method comprising, or consisting essentially of, or yet further consisting of, administering to the subject an effective amount of a recombinant peptide described herein or an FBP1 E7 fragment or an equivalent of each thereof.

Further aspects of the compounds, compositions and methods are disclosed herein.

### BRIEF DESCRIPTION OF THE DRAWINGS

FIGS. 1A-1T: Hepatomegaly, hepatosteatosis, hypoglycemia in fasted Fbp1^{ΔHep} mice. (FIG. 1A) Gross morphology of livers from 8-weeks-old (wo) Fbp1^{F/F} and Fbp1^{ΔHep} mice fasted for 16 h. (FIG. 1B) Hematoxylin and eosin (H&E), Oil Red O (ORO), and periodic acid Schiff (PAS) staining of liver sections from 1A (n=8). Scale bars, 20 μm. (FIG. 1C) ORO and PAS staining intensity per high-magnification-field (HMF) was determined by Image J from the slides shown in 1B. (FIGS. 1D-1H) Liver/Body weight (FIG. 1D), liver triglycerides (TG) (FIG. 1E), and serum glucose (FIG. 1F), TG (FIG. 1G), and cholesterol (FIG. 1H) in the indicated mice (n=9-10). (FIG. 1I and FIG. J) Serum aspartate aminotransferase (AST)/alanine aminotransferase (ALT) ratio (FIG. 1I) and liver NADPH/NADP+ ratio (FIG. 1J) in the indicated mice (n=6). (FIG. 1K) Gross morphology of livers from 8-weeks-old (wo) Fbp1^{F/F} and Fbp1^{ΔHep} mice fasted for 4 h. (FIG. 1L) H&E, ORO, and PAS staining of liver sections from K (n=7). Scale bars, 20 μm. (FIG. 1M) ORO and PAS staining intensity per HMF was determined by Image J analysis of the data in 1L. (FIGS. 1N-1R) Liver/Body weight (FIG. 1N), liver TG (FIG. 1O), and serum glucose (FIG. 1P), TG (FIG. 1Q), and cholesterol (FIG. 1R) in the indicated mice (n=7). (FIG. 1S and FIG. 1T) Serum AST/ALT ratio (FIG. 1S) and liver NADPH/NADP+ ratio (FIG. 1T) in the indicated mice (n=7). Data are presented as mean±SEM. *P<0.05, **P<0.01, ***P<0.001, ****p<0.0001 (Unpaired two-tailed t test).

FIGS. 2A-2O: Fasted Fbp1^{ΔHep} mice exhibit enhanced AKT-mTOR signaling and elevated de novo lipogenesis (FIG. 2A) Immunoblot (IB) analysis of the indicated proteins in livers of 16 h fasted 8-wo Fbp1^{F/F} and Fbp1^{ΔHep} mice. Densitometric quantification of relative normalized protein ratios (Fbp1^{ΔHep}/Fbp1^{F/F}) and P values are shown on the right. (FIG. 2B) IB analysis of indicated nuclear and cytoplasmic proteins in 16 h fasted 8-wo Fbp1^{F/F} and Fbp1^{ΔHep} mice (n=4). Relative normalized protein ratios (Fbp1^{ΔHep}/Fbp1^{F/F}) and P values are shown on the right. (FIGS. 2C-2G) qRT-PCR analysis of lipogenesis (FIG. 2C), cholesterol synthesis (FIG. 2D), lipid uptake (FIG. 2E), glycolysis, pentose phosphate pathway, glycogen synthesis (FIG. 2F) and β-oxidation (FIG. 2G) related liver mRNAs from fasted 8-wo Fbp1^{F/F} and Fbp1^{ΔHep} mice (n=9-10). (FIGS. 2H-2J) Fractional labeling of liver F6-P (FIG. 2H), G6-P (FIG. 2I) and amino acids (FIG. 2J) from 13C-lactate tracing of 16 h fasted 8-wo Fbp1^{F/F} and Fbp1^{ΔHep} mice (n=5). (FIG. 2K and FIG. 2L) Relative deuterium incorporation into newly synthesized liver palmitate (C16:0) (FIG. 2K) and stearate (C18:0) (FIG. 2L) after 24 h of D2O labeling (n=4-6). (FIG. 2M) IB analysis of indicated liver proteins from 4 h fasted 8-wo Fbp1^{F/F} and Fbp1^{ΔHep} mice. Relative normalized protein ratios (Fbp1^{ΔHep}/Fbp1^{F/F}) and P values are shown on the right. (FIG. 2N) qRT-PCR analysis of lipogenesis related liver mRNAs from 4 h fasted 8-wo Fbp1^{F/F} and Fbp1^{ΔHep} mice (n=7). (FIG. 2O) Liver protein synthesis in 4 h fasted 8-wo Fbp1^{F/F} and Fbp1^{ΔHep} mice was imaged (left) by O-propargyl-puromycin (OPP, TMR-azide) incorporation (n=5), and mean fluorescence intensity was quantified (right). Scale bars, 20 μm. Data are presented as mean±SEM. *P<0.05, **P<0.01, ***P<0.001, ****p<0.0001 (Unpaired two-tailed t test). All blots are representative of at least 3 biological replicates.

FIGS. 3A-3I: AKT inhibition attenuate hepatomegaly, hepatosteatosis and hyperlipidemia but not hypoglycemia. (FIG. 3A) Gross liver morphology of 8-wo Fbp1^{F/F} and Fbp1^{ΔHep} mice treated −/+MK2206 (100 mg/kg) or Torin1 (10 mg/kg), followed by 16 h fast before sacrifice. (B-D) (FIGS. 3B-3D) H&E (FIG. 3B) and ORO staining (FIG. 3C) of liver sections from 3A (n=4-5). Scale bars, 20 μm. ORO staining intensity per HMF was determined by Image J (FIG. 3D). (FIGS. 3E-3I) Liver TG (FIG. 3E), serum TG (FIG. 3F), serum glucose (FIG. 3G), serum cholesterol (FIG. 3H) and liver/body weight ratio (FIG. 3I) in indicated mice (n=4-5). Data are presented as mean±SEM. n.s., not significant, P≥0.05. *P<0.05, **P<0.01, ***P<0.001, ****p<0.0001 (Unpaired two-tailed t test).

FIGS. 4A-4H: Inactive FBP1 prevents hepatomegaly, steatosis, and hyperlipidemia. (FIG. 4A) Gross liver morphology of 8-wo Fbp1^{F/F} and Fbp1^{ΔHep} transduced with AAV8-Ctrl, AAV8-FBP1 and AAV8-FBP1^{E98A}, followed by 16 h fast before sacrifice at 3 wk post-AAV8 infection (n=4-5). (FIG. 4B) Liver/body weight ratio in above mice (n=4-5). (FIG. 4C and FIG. 4D) H&E and ORO staining of liver sections from 4A (n=4-5) (FIG. 4C). Scale bars, 20 μm. ORO staining intensity per HMF determined by Image J (FIG. 4D). (FIGS. 4E-4H) liver TG (FIG. 4E), serum TG (FIG. 4F), serum cholesterol (FIG. 4G), and serum glucose (FIG. 4H) in the indicated mice (n=4-5). Data are mean±SEM. n.s., not significant, P≥0.05, *P<0.05, **P<0.01, ***P<0.001 (Unpaired two-tailed t test).

FIGS. 5A-5M: FBP1 associates with PP2A-C and ALDOB to inhibit AKT activation. (FIG. 5A) IP analysis of liver lysates from Fbp1^{F/F} and Fbp1^{ΔHep} mice transduced with AAV8-Ctrl, AAV8-FBP1 and AAV8-FBP1^{E98A} (n=3). The gel separated IPs were IB'ed with the indicated antibodies. (FIG. 5B) IP of primary mouse hepatocytes isolated from 16 h fasted Fbp1^{F/F} and Fbp1^{ΔHep} mice (n=3). The gel separated IPs were IB'ed with the indicated antibodies. (FIG. 5C) IP analysis of liver lysates from fed or 16 h fasted 8-wo Fbp1^{F/F} and Fbp1^{ΔHep} mice. The gel separated IPs were IB'ed with the indicated antibodies. (FIGS. 5D-5F) In vitro associations between FBP1 (FIG. 5D), ALDOB (FIG. 5E), and GST-PP2A-C (FIG. 5F) with the indicated recombinant proteins. The gel separated IPs were IB'ed with the indicated antibodies. (FIG. 5G and FIG. 5H) PLA of AKT-FBP1 (FIG. 5G, top) and AKT-PP2A-C (FIG. 5H, left) interactions in liver sections of fed or 16 h fasted 8-wo Fbp1^{F/F} and Fbp1^{ΔHep} mice (n=5-8). Scale bars, 10 μm. Quantification of representative stained tissues (FIG. 5G, bottom and FIG. 5H, right). (FIG. 5I) IP of liver lysates of WT mice treated −/+ insulin (0.5 U/kg) after an overnight fast (n=3). The gel separated IP's were IB'ed with indicated antibodies. (FIG. 5J and FIG. 5K) IB analysis of Huh7 cells stably transfected with the indicated vectors and stimulated or not with insulin (100 nM) for 1 h after 6 h of serum starvation. (FIG. 5L and FIG. 5M) In vitro dephosphorylation of phosphorylated HA-AKT1 isolated from insulin stimulated Huh7 cells and incubated with active-PP2A-C in the presence or absence of indicated recombinant proteins. The reactions were IB analyzed with the indicated antibodies (FIG. 5L). Relative intensity of P-AKT (S473) and P-AKT (T308) was determined by densitometry (FIG. 5M). Data are presented as mean±SEM. *P<0.05, **P<0.01, ***P<0.001 (Unpaired two-tailed t test).

FIGS. 6A-6M: Fbp1^{ΔHep} mice and FBP1-deficient human hepatocytes are insulin hyperresponsive. (FIG. 6A) Gross liver morphology of 18-wo HFD-fed Fbp1^{F/F} and IFbp1^{ΔHep} mice (n=8-10) that were fasted for 4 h before sacrifice. (FIG. 6B) H&E, ORO, and PAS staining of liver sections from above mice (n=8). Scale bars, 20 μm. (FIG. 6C) ORO and PAS staining intensity per HMF from 6C. (FIGS. 6D-6G) Serum glucose (FIG. 6D), liver TG (FIG. 6E), serum TG (FIG. 6F) and serum cholesterol (FIG. 6G) in indicated HFD-fed mice (n=8-10). (FIG. 6H) Glucose tolerance test (GTT) of HFD-fed Fbp1^{F/F} and Fbp1 ^{ΔHep} mice (left) (n=3) and area under the curve (AUC) quantification (right). (FIG. 6I) Insulin tolerance test (ITT) of HFD-fed Fbp1^{F/F} andFbp1^{ΔHep} mice (left) (n=4) and AUC quantification (right). (FIG. 6J) IB analysis of liver lysates from indicated HFD-fed mice. Densitometric quantification of relative normalized protein ratios (Fbp1^{ΔHep}/Fbp1^{F/F}) and P values are shown on the right. (FIG. 6K) IB analysis of liver lysates of HFD-fed 18-wo Fbp1^{F}/F and Fbp1^{ΔHep} mice collected 15 min after control or insulin injections (n=4). Quantification of relative normalized protein amounts is shown below each P-AKT strip. (FIG. 6L) ITT of NCD-fed Fbp1^{F/F} and Fbp1^{ΔHep} mice (top) (n=4-5) and AUC quantification (bottom). (FIG. 6M) Human hepatocytes stably transfected with shCtrl or shFBP1 were stimulated with the indicated concentrations of insulin for 1 h after 6 h of serum starvation and IB analyzed with the indicated antibodies. Data are mean±SEM. *P<0.05, **P<0.01, ***P<0.001, ****p<0.0001 (Unpaired two-tailed t test).

FIGS. 7A-7J: Complex-disrupting FBP1-derived C-terminal peptide ameliorates insulin resistance (FIG. 7A) IB analysis of Huh7 cells stably expressing V5-tagged human FBP1 missense mutants incubated −/+ insulin (100 nM, 1 h) after 6 h serum starvation. (FIG. 7B) V5-FBP1, V5-FBP1^{E98A} and V5-FBP^{L129P} were IP'ed from stably transfected Huh7 cells and the gel separated IPs were IB'ed with the indicated antibodies. (FIG. 7C) To map FBP1 regions interacting with AKT, PP2A-C and ALDOB, V5-tagged FBP1 exon deletion mutants were stably expressed in Huh7 cells and their association with AKT, PP2A-C and ALDOB was examined by IP with V5 antibody. (FIG. 7D and FIG. 7E) Male BL6 mice were placed on HFD for 14 w to induce insulin resistance. The mice were injected every other day with Ctrl or FBP1 E7 peptides (10 mg/kg each) for 2 w. The mice were then fasted overnight before analysis. Liver lysates were IP'ed with FBP1 (FIG. 7D) or AKT (FIG. 7E) antibodies and AKT, FBP1 and PP2A-C co-IPs were IB analyzed. (FIG. 7F) The GST-PP2A-C pull down of indicated recombinant proteins was performed in the presence of FBP1 E7 or Ctrl peptides and analyzed as above. (FIG. 7G) HFD-fed BL6 mice from 7D were injected −/+ insulin 15 min before being analyzed for liver AKT activation. Relative normalized protein amounts are indicated below the P-AKT strips. (FIG. 7H) Mice from 7D were fasted overnight and subjected to GTT (n=5) (left) and AUC quantification (right). (FIG. 7I) ITT of mice from 7D that were fasted for 2-4 h (n=5) (FIG. 7I, left) and AUC quantification (FIG. 7I, right). (FIG. 7J) AST/ALT ratio in mice from 7D (n=5). Data are presented as mean±SEM. *P<0.05, **P<0.01, ***P<0.001 (Unpaired two-tailed t test).

FIGS. 8A-8W: Hepatocyte specific FBP1 ablation increases ATP, acetyl-CoA and serum lactate while decreasing glycogen and G6-P amounts after 16 h and 4 h fast, Related to FIG. 1. (FIG. 8A) Schematic representation of glucose metabolism and roles of FBP1 and ALDOB in GNG. (FIG. 8B) Epididymal white adipose tissue (eWAT), subcutaneous adipose tissue (SQ-WAT), brown adipose tissue (BAT), kidney and spleen weight/body weight ratio in 16 h fasted 8-wo Fbp1^{F/F} and Fbp1^{ΔHep} mice (n=9-10). (FIG. 8C and FIG. 8D) Nuclei per field (FIG. 8C) and hepatocyte DNA content (FIG. 8D) from 16 h fasted 8-wo Fbp1^{F/F} and Fbp1^{ΔHep} livers (n=6-9). (FIG. SE) qRT-PCR analysis of lipolytic mRNAs in eWAT of 16 h fasted 8-wo Fbp1^{F/F} and Fbp1^{ΔHep} mice (n=9-10). (FIGS. 8F-8H) NADPH (FIG. 8F), ATP (FIG. 8G) and acetyl-CoA (FIG. 8H) concentrations of liver lysates from above mice (n=6-8). (FIG. 8I and FIG. 8J) Serum insulin (FIG. 8I) and glucagon (FIG. 8J) in indicated mice (n=6-8). (FIGS. 8K-8M) Serum lactate (FIG. 8K), liver glycogen (FIG. 8L) and G6-P (FIG. 8M) in indicated mice (n=9-10). (FIG. 8N and FIG. 8O) NADPH (FIG. 8N) and ATP (FIG. 8O) concentrations in liver lysates of 4 h fasted 8-wo Fbp1^{F/F} and Fbp1^{ΔHep} mice (n=7). (FIG. 8P) eWAT, SQ-WAT, BAT, kidney, and spleen weight/body weight ratio in 4 h fasted 8-wo Fbp1^{F/F} and Fbp1^{ΔHep} mice (n=7). (FIG. 8Q) qRT-PCR of lipolytic mRNAs in eWAT of 4 h fasted 8-wo Fbp1^{F/F} and Fbp1^{ΔHep} mice (n=7). (FIG. 8R) IB analysis of indicated proteins in eWAT of 4 h fasted 8-wo Fbp1^{F/F} and Fbp1^{ΔHep} mice. Densitometry determined relative normalized protein ratios (Fbp1^{ΔHep}/Fbp1^{F/F}) and P values are shown on the right. (FIGS. 8S-8U) Serum lactate (FIG. 8S), insulin (FIG. 8T) and glucagon (FIG. 8U) in the indicated mice (n=7). (FIG. 8V and FIG. 8W) Liver glycogen (FIG. 8V) and G6-P (FIG. 8W) in the indicated mice (n=7). Data are presented as mean±SEM. *P<0.05, **P<0.01, ***P<0.001, ****p<0.0001 (Unpaired two-tailed t test). n.s., not significant.

FIGS. 9A-9M: Fasting induced metabolic genes and metabolic alterations in Fbp1^{ΔHep} mice, Related to FIG. 2. (FIG. 9A) IB analysis of mouse primary hepatocytes from fasted 8-wo Fbp1^{F/F} and Fbp1^{ΔHep} mice. Relative normalized protein ratios (Fbp1^{ΔHep}/Fbp1^{F/F}) and P values are shown on the right. (FIG. 9B) IB analysis of nuclear and cytosolic liver proteins in 16 h fasted 8-wo Fbp1^{F/F} and Fbp1^{ΔHep} mice. Relative normalized protein ratios (Fbp1^{ΔHep}/Fbp1^{F/F}) and P values are shown on the right. (FIG. 9C) Schematic of U-¹³C glucose and ¹³C-Lactate tracing of 16 h fasted 8-wo Fbp1^{F/F} and Fbp1^{ΔHep} livers (n=4-6). (FIG. 9D and FIG. 9E) Fractional labelling of glycolytic (FIG. 9D) and TCA (FIG. 9E) metabolites from U-¹³C glucose tracing of fasted 8-wo Fbp1^{F/F} and Fbp1 ^{ΔHep} liver (n=4-6). (FIG. 9F) Fractional labelling of liver TCA intermediates from ¹³C-lactate tracing of indicated livers (n=5). (FIG. 9G) P-GS(S641) and GYS2 in liver lysates of fasted 8-wo Fbp1^{F/F} and Fbp1^{ΔHep} mice. Relative normalized relative GYS2 and P-GYS2 ratios (Fbp1^{ΔHep}/Fbp1^{F/F}) and P values are shown on the right. (FIG. 9H) Glycogen synthase activity in lysates of 16 h fasted 8-wo Fbp1^{F/F} and Fbp1^{ΔHep} livers (−/+) saturating exogenous G6-P (n=7-8). (FIGS. 9I-9L) qRT-PCR of cholesterol synthesis (FIG. 9I), lipid uptake (FIG. 9J), glycolysis, pentose phosphate pathway, glycogen synthesis (FIG. 9K) and β-oxidation (FIG. 9L) related mRNAs in livers of 4 h fasted 8-wo Fbp1^{F/F} and Fbp1^{ΔHep} mice (n=7). (FIG. 9M) IB analysis of nuclear and cytosolic extracts of 4 h fasted 8-wo Fbp1^{F/F} and Fbp1^{ΔHep} livers. Relative normalized protein ratios (Fbp1^{ΔHep}/Fbp1^{F/F}) and P values are shown on the right. Data are presented as mean±SEM, *P<0.05, **P<0.01, ***P<0.001, ****p<0.0001, n.s., not significant. (Unpaired two-tailed t test).

FIGS. 10A-10U: The fed Fbp1^{ΔHep} liver is metabolically normal, Related to FIG. 2. (FIG. 10A) Gross morphology of livers from 8-weeks-old (wo) Fbp1^{F/F} and Fbp1^{ΔHep} mice at the fed (ad libitum) state (n=9-10). (FIG. 10B) H&E, ORO, and PAS staining of liver sections from the mice in S3A. Scale bars, 20 μm. (FIG. 10C) ORO and PAS staining intensity per HMF were determined by Image J analysis of the liver sections in S3B. (FIGS. 10D-10M) Liver/body weight (FIG. 10D), nuclei per field (FIG. 10E), hepatocyte DNA content (FIG. 10F), and liver TG (FIG. 10G), acetyl-CoA (FIG. 10H) and glycogen (FIG. 10I), and serum AST/ALT ratio (FIG. 10J), glucose (FIG. 10K), TG (FIG. 10L), and cholesterol (FIG. 10M) in the indicated mice (n=6-10). (FIG. 10N and FIG. 10O) Serum insulin (FIG. 10N) and glucagon (FIG. 10O) in the indicated mice (n=6-10). (FIGS. 10P-10T) qRT-PCR of lipogenesis (FIG. 10P), cholesterol synthesis (FIG. 10Q), lipid uptake (FIG. 10R), glycolysis, pentose phosphate pathway, glycogen synthesis (FIG. 10S) and p-oxidation (FIG. 10T) related mRNAs in livers of fed 8-wo Fbp1^{F/F} and Fbp1^{ΔHep} mice (n=9-10). (FIG. 10U) P-GS(S641) and GYS2 in liver lysates of fed 8-wo Fbp1^{F/F} and Fbp1^{ΔHep} mice. Relative normalized protein ratios (Fbp1^{ΔHep}/Fbp1^{F/F}) and P values are shown on the right. Data are presented as mean±SEM, *P<0.05, ***P<0.001, n.s., not significant. (Unpaired two-tailed t test).

FIGS. 11A-11E: AKT and mTORC inhibitors attenuate hepatomegaly, hepatosteatosis and hyperlipidemia in fasted Fbp1^{ΔHep} mice, Related to FIG. 3. (FIGS. 11A-11B) Liver protein synthesis in untreated or MK2206 or Torin 1 pretreated and 16 h fasted 8-wo Fbp1^{F/F} and Fbp1^{ΔHep} mice was determined by OP-puro (TMR-azide) incorporation (FIG. 11A) (n=4-5). Scale bars, 20 μm. Mean fluorescence intensity of above liver sections (FIG. 11B). (FIG. 11C) Serum insulin levels in the mice from FIG. 3A (n=4-5). (FIG. 11D) IB analysis of liver lysates from above mice (left) and relative normalized protein amounts (right). (FIG. 11E) qRT-PCR of liver Srebplc, Acly, Fasn, Hmgcr and Cd36 mRNAs in the mice from FIG. 3A (n=4-5). Data are presented as mean±SEM. *P<0.05, **P<0.01, ***P<0.001, ****p<0.0001, n.s., not significant. (Unpaired two-tailed t test).

FIGS. 12A-12E: Catalytically inactive FBP1 restrains hepatomegaly, fatty liver, and hyperlipidemia, but not hypoglycemia in Fbp1^{ΔHep} mice, Related to FIG. 4. (FIG. 12A and FIG. 12B) qRT-PCR of liver Acly, Acc1, Fiasn (FIG. 12A), Hmgcs1 and Hmgcr (FIG. 12B) mRNAs of fasted Fbp1^{F/F} and Fbp1^{ΔHep} mice transduced with AAV8-Ctrl, AAV8-FBP1 and AAV8-FBP1^{E98A} (n=4-5). (FIG. 12C and FIG. 12D) IB analysis of liver lysates from the mice in FIG. 4A (FIG. 12C) and relative normalized protein amounts (FIG. 12D). (FIG. 12E) Scatterplots of FBP1 interacting proteins identified by MS analysis of HA IPs from Fbp1^{ΔHep} livers transduced with AAV8-HA-control or AAV8-HA-FBP1. The liver lysates were immuno-purified on anti-HA affinity beads. The eluates were washed and analyzed by mass spectrometry. Data are presented as mean±SEM. *P<0.05, **P<0.01, ***P<0.001, ****p<0.0001, n.s., not significant. (Unpaired two-tailed t test).

FIGS. 13A-13W: FBP1 associates with ALDOB and PP2A-C to form a protein complex that binds AKT and inhibits its activation, Related to FIG. 5. (FIG. 13A) IP analysis of 293T cells transfected with Ctrl, V5-FBP1 or V5-FBP1^{E98A} vectors. The gel separated IPs were IB'ed with the indicated antibodies. (FIG. 13B) IP analysis of 293T cells transfected with empty Flag or Flag-ALDOB vectors. The gel separated IPs were IB'ed with the indicated antibodies. (FIG. 13C) IP analysis of 293T cells transfected with HA-AKT1, V5-FBP1 and Flag-PP2A-C. The gel separated IPs were IB'ed with the indicated antibodies. (FIG. 13D) IP analysis of Huh7 cells stably transfected with FBP1, FBP1^{E98A} or Ctrl vectors. The gel separated IPs were IB'ed with the indicated antibodies. (FIG. 13E and FIG. 13F) IP analysis with indicated antibodies of Huh7 cells stably transfected with shCtrl, shFBP1 (FIG. 13E) and sgCtrl or sgALDOB (FIG. 13F). The gel separated IPs were IB'ed with the indicated antibodies. (FIG. 13G and FIG. 13H) IP analysis of liver lysates from 8-wo Fbp1^{F/F} and Fbp1^{ΔHep} mice fed or fasted for 16 h (FIG. 13G) or 4h (FIG. 13H). The gel separated IPs were IB'ed with the indicated antibodies. (FIG. 13I and FIG. 13J) Liver lysates of fasted 8-wo Fbp1^{F/F} and Fbp1^{ΔHep} mice were analyzed by gel filtration chromatography. Fractions were resolved by SDS-PAGE and probed with the indicated antibodies (FIG. 13I). Fractions 2-14 were analyzed again as above (FIG. 13J). Arrows indicate the approximate elution volumes of molecular weight standards: blue dextran, Vo; thyroglobulin, 670 kDa; γ-globulin, 158 kDa; ovalbumin, 44 kDa; and myoglobin, 17 kDa. (FIG. 13K and FIG. 13L) Quantification of representative PLA images (n=5-12) of ALDOB-FBP1 (FIG. 13K) and PP2A-C-FBP1 (FIG. 13L) interactions determined with liver sections of fed or fasted (16 h) 8-wo WT mice stained with the indicated antibody combinations. (FIG. 13M and FIG. 13N) Quantification of representative images of ALDOB-AKT (FIG. 13M) and ALDOB-PP2A-C (FIG. 13N) interactions detected by PLA of liver sections of fed or 16 h fasted 8-wo Fbp1^{F/F} and Fbp1^{ΔHep} mice stained with the indicated antibody combinations. (FIGS. 130-13T) Quantification of representative PLA images of AKT-FBP1 (FIG. 13O), ALDOB-FBP1 (FIG. 13P), PP2A-C-FBP1 (FIG. 13), AKT-PP2A-C (FIG. 13R), AKT-ALDOB (FIG. 13S), and ALDOB-PP2A-C (FIG. 13T) interactions detected by PLA of liver sections from fasted 8-wo WT mice treated −/+ insulin (0.5U/kg) stained with the indicated antibody combinations. (FIG. 13U) HK-2 and Caco-2 cells stably transfected with shCtrl or shFBP1 were treated −/+ insulin (100 nM) and IB'ed with indicated antibodies. (FIG. 13V) In vitro dephosphorylation of HA-AKT1 isolated from insulin stimulated Huh7 cells and incubated with active-PP2A-C in the presence or absence of ALDOB. The reactions were JIB analyzed with the indicated antibodies (left). Relative P-AKT (S473) and P-AKT (T308) intensity was determined by densitometry (right). (FIG. 13W) HA-c-Myc was isolated from EGF (100 ng/ml) stimulated stably expressing Huh7 cells incubated with active PP2A-C in the presence or absence of recombinant FBP1 and ALDOB. The reactions were IB analyzed with the indicated antibodies (left). Relative c-Myc (p-S62) intensity was determined by densitometry (right). Data are presented as mean±SEM. *P<0.05, **P<0.01, ***P<0.001, ****p<0.0001 (Unpaired two-tailed t test).

FIGS. 14A-14Y: Fbp1^{ΔHep} mice are insulin hyperresponsive, Related to FIG. 6. (FIG. 14A) IP analysis of liver lysates from 18-wo NCD and HFD fed WT mice. The gel separated IPs were 1IB'ed with the indicated antibodies. (FIGS. 14B-14D) Representative images of AKT-FBP1 (FIG. 14B, left), ALDOB-FBP1 (FIG. 14C, left) and PP2A-C-FBP1 (FIG. 14D, left) interactions detected by PLA of liver sections of NCD or HFD fed WT mice stained with the indicated antibody combinations. Scale bars, 10 μm. Quantification of the PLA signals is shown on the right. (FIG. 14E) Body weight gain by HFD-fed Fbp1^{F/F} and Fbp1^{ΔHep} mice (n=9-10). (FIG. 14F) Liver/body weight ratio in indicated HFD-fed mice (n=9-10). (FIG. 14G) Insulin levels during the GTT shown FIG. 6H (n=3) (left) and AUC quantification (right). (FIG. 14H) IB analysis of NCD-fed 8-wo Fbp1^{F/F} and Fbp1^{ΔHep} liver lysates prepared 15 min after control or insulin injections. Relative normalized protein amounts are shown below the P-AKT strips. (FIG. 14I and FIG. 14J) GTT (FIG. 141, left) and ITT (FIG. 14J, left) performed on fasted Fbp1^{F/F} and Fbp1^{ΔHep} mice transduced with AAV8-Ctrl, AAV8-FBP1 or AAV8-FBP1^{E98A} (n=4-5). AUC quantification of glucose amounts (FIG. 14I and FIG. 14J, right). (FIG. 14K and FIG. 14L) Insulin concentrations during GTT of the indicated mice (FIG. 14K) (n=4-5) and AUC quantification (FIG. 14L). (FIG. 14M and FIG. 14N) Pyruvate tolerance test (PTT) performed on fasted Fbp1^{F/F} and Fbp1^{ΔHep} mice transduced with AAV8-Ctrl, AAV8-FBP1 or AAV8-FBP1^{E98A} (n=4-5) (FIG. 14M). AUC quantification of glucose amounts (FIG. 14N). (FIG. 14O) Human hepatocytes stably transfected with shCtrl or shFBP1 were stimulated with insulin (100 nM) for the indicated times after 6 h serum starvation. The cells were lysed and IB analyzed with the indicated antibodies. (FIG. 14P and FIG. 14Q) Serum insulin (FIG. 14P) and glucose (FIG. 14Q) of −/+STZ treated Fbp1^{F/F} and Fbp1^{ΔHep} mice (n=4-7). (FIG. 14R) Gross liver morphology of 4 h fasted STZ treated 8-wo Fbp1^{F/F} and Fbp1^{ΔHep} mice (n=4-6). (S-U) (FIGS. 14S-14U) Liver/body weight ratio (FIG. 14S), serum TG (FIG. 14T) and cholesterol (FIG. 14U) of STZ treated Fbp1^{F/F} and Fbp1^{ΔHep} mice from S7R (n=4-6). (FIG. 14V) IB analysis of liver lysates of STZ treated and fasted Fbp1^{F/F} and Fbp1^{ΔHep} mice. Relative normalized protein ratios (Fbp1^{ΔHep}/Fbp1^{F/F}) and P values are shown on the right. (FIGS. 14W-14Y) qRT-PCR ofAcly, Acc1, Fasn (FIG. 14W), Hmgcs1, Hngcr (FIG. 14X) and Cd36 (FIG. 14Y) mRNAs in livers of STZ treated and fasted Fbp1^{F/F} and Fbp1^{ΔHep} mice (n=4-6). Data are presented as mean±SEM. *P<0.05, **P<0.01, ***P<0.001, ****p<0.0001, n.s., not significant (Unpaired two-tailed t test).

FIGS. 15A-15W: An FBP1 C-terminal peptide that disrupts FBP1:AKT:PP2A-C interactions activates AKT and ameliorates insulin resistance, Related to FIG. 7. (FIG. 15A) Normalized FBP1 enzyme activities of WT and missense human FBP1 mutants expressed in Huh7 cells. (FIG. 15B) Schematic of FBP1 exon deletion mutants. (FIG. 15C) Huh7 cells stably expressing the indicated proteins were treated with −/+ insulin (100 nM) and IB'ed with the indicated antibodies. (FIG. 15D) The sequence of the cell permeable E7 peptide (preceded by the TAT peptide) corresponding to FBP1 AA 275-300. The scrambled peptide was used as a control. (FIGS. 15E-15G) AlphaFold predictions of E7 peptide docking onto AKT1 (FIG. 15E), PP2A-C (FIG. 15F) and ALDOB (FIG. 15G). (FIGS. 15H-15J) Relative FBP1 mRNA levels according to GEO databases GSE15653 of livers from lean, obese and obese T2DM (FIG. 15H), GSE64998 of livers from non-obese and obese individuals (FIG. 15I), GSE64998 of livers from non-obese, ND-obese and T2D-obese individuals (FIG. 15J). T2DM, Type 2 diabetes. ND-obese, non-diabetic obese. (FIGS. 15K-15N) Representative images of AKT-PP2A-C (FIG. 15K, left), AKT-FBP1 (FIG. 15L, left), FBP1-PP2A-C (FIG. 15M, left) and FBP1-ALDOB (FIG. 15N, left) interactions detected by PLA of liver sections of HFD-fed WT mice treated with Ctrl or FBP1 E7 peptides and stained with the indicated antibody combinations. Scale bars, 10 μm. Quantification of representative PLA images is shown on the right. (FIG. 15O) In vitro dephosphorylation of HA-AKT1 isolated from insulin stimulated Huh7 cells and incubated with active-PP2A-C in the presence or absence the E7 peptide. The reactions were IB analyzed with the indicated antibodies (left). Relative intensity of P-AKT (S473) and P-AKT (T308) was determined by densitometry (right). (FIG. 15P) Insulin levels during the GTT from FIG. 7H (left) and AUC quantification of the insulin levels (right) (n=5). (FIG. 15Q) PTT performed on the HFD-fed mice from FIG. 7D fasted for 12-14h (left) and AUC quantification (right). (FIG. 15R) Serum glucose in mice from FIG. 7D (n=5). (FIG. iSS and FIG. 15T) ORO and PAS staining of liver sections from FIG. 7D (FIG. 15S). Scale bars, 20 μm. ORO and PAS staining positive areas per HMF (FIG. 15T). (FIG. 15U) Serum TG in mice from FIG. 7D (n=5). (FIG. 15V) Body weight gain of HFD fed mice treated with Ctrl and FBP1 E7 peptide (n=5). (FIG. 15W) Normalized FBP1 activity in HFD fed mice from FIG. 7D (n=5). Data are presented as mean±SEM. *P<0.05, **P<0.01, ***P<0.001, n.s., not significant (Unpaired two-tailed t test).

### DETAILED DESCRIPTION OF THE DISCLOSURE

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

Throughout this disclosure, various publications, patents and published patent specifications may be referenced by an identifying citation or by an Arabic numeral or first author name. The full citation for the publications identified by an Arabic numeral or first author name are found immediately preceding the claims. The disclosures of these publications, patents and published patent specifications are hereby incorporated by reference into the present disclosure in their entirety to more fully describe the state of the art to which this disclosure pertains.

The practice of the present technology will employ, unless otherwise indicated, conventional techniques of organic chemistry, pharmacology, immunology, molecular biology, microbiology, cell biology and recombinant DNA, which are within the skill of the art. See, e.g., Sambrook, Fritsch and Maniatis, Molecular Cloning: A Laboratory Manual, 2nd edition (1989); Current Protocols In Molecular Biology (F. M. Ausubel, el al. eds., (1987)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, a Laboratory Manual, and Animal Cell Culture (R. I. Freshney, ed. (1987)).

The term “about,” as used herein when referring to a measurable value such as an amount or concentration and the like, is meant to encompass variations of 20%, 10%, 5%, 1%, 0.5%, or even 0.1% of the specified amount.

The terms or “acceptable,” “effective,” or “sufficient” when used to describe the selection of any components, ranges, dose forms, etc. disclosed herein intend that said component, range, dose form, etc. is suitable for the disclosed purpose.

Also as used herein, “and/or” refers to and encompasses any and all possible combinations of one or more of the associated listed items, as well as the lack of combinations when interpreted in the alternative (“or”). “Optional” or “optionally” means that the subsequently described circumstance may or may not occur, so that the description includes instances where the circumstance occurs and instances where it does not.

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

As used herein, a biological sample, or a sample, is obtained from a subject. Exemplary samples include, but are not limited to, cell sample, tissue sample, tumor biopsy, liquid samples such as blood and other liquid samples of biological origin (including, but not limited to, ocular fluids (aqueous and vitreous humor), peripheral blood, sera, plasma, ascites, urine, cerebrospinal fluid (CSF), sputum, saliva, bone marrow, synovial fluid, aqueous humor, amniotic fluid, cerumen, breast milk, broncheoalveolar lavage fluid, semen, prostatic fluid, cowper's fluid or pre-ejaculatory fluid, female ejaculate, sweat, tears, cyst fluid, pleural and peritoneal fluid, pericardial fluid, ascites, lymph, chyme, chyle, bile, interstitial fluid, menses, pus, sebum, vomit, vaginal secretions/flushing, synovial fluid, mucosal secretion, stool water, pancreatic juice, lavage fluids from sinus cavities, bronchopulmonary aspirates, blastocyl cavity fluid, or umbilical cord blood.

As used in the specification and claims, the singular form “a”, “an” and “the” include plural references unless the context clearly dictates otherwise. For example, the term “a cell” includes a plurality of cells, including mixtures thereof.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this disclosure or process steps to produce a composition or achieve an intended result. Embodiments defined by each of these transition terms are within the scope of this disclosure.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

The term “purified” refers to a composition being substantially free from contaminants. With respect to polynucleotides and polypeptides, purified intends the composition being substantially free from contamination from polynucleotides or polypeptides with different sequences. In certain embodiments, it also refers to polynucleotides and polypeptides substantially free from cell debris or cell culture media.

The term “recombinant” refers to a form of artificial DNA that is created by combining two or more sequences that would not normally occur in their natural environment. A recombinant protein is a protein that is derived from recombinant DNA.

The term “binding” or “binds” as used herein are meant to include interactions between molecules that may be covalent or non-covalent which, in one embodiment, can be detected using, for example, a hybridization assay. The terms are also meant to include “binding” interactions between molecules. Interactions may be, for example, protein-protein, antibody-protein, protein-nucleic acid, protein-small molecule or small molecule-nucleic acid in nature. This binding can result in the formation of a “complex” comprising the interacting molecules. A “complex” refers to the binding of two or more molecules held together by covalent or non-covalent bonds, interactions or forces.

The term “polypeptide” is used interchangeably with the term “protein” and “peptide” and in its broadest sense refers to a compound of two or more subunit amino acids, amino acid analogs or peptidomimetics. The subunits may be linked by peptide bonds. In another embodiment, the subunit may be linked by other bonds, e.g., ester, ether, etc. As used herein the term “amino acid” refers to natural and/or unnatural or synthetic amino acids, including glycine and both the D and L optical isomers, amino acid analogs and peptidomimetics. A peptide of three or more amino acids is commonly called an oligopeptide if the peptide chain is short. If the peptide chain is long, the peptide is commonly called a polypeptide or a protein. The term “peptide fragment,” as used herein, also refers to a peptide chain.

The phrase “biologically equivalent polypeptide” or “biologically equivalent peptide fragment” refers to protein, polynucleotide, or peptide fragment which hybridizes to the exemplified polynucleotide or peptide fragment under stringent conditions and which exhibit similar biological activity in vivo, e.g., approximately 100%, or alternatively, over 90% or alternatively over 85% or alternatively over 70%, as compared to the standard or control biological activity. Additional embodiments within the scope of this disclosure are identified by having more than 60%, or alternatively, more than 65%, or alternatively, more than 70%, or alternatively, more than 75%, or alternatively, more than 80%, or alternatively, more than 85%, or alternatively, more than 90%, or alternatively, more than 95%, or alternatively more than 97%, or alternatively, more than 98% or 99% sequence homology. Percentage homology can be determined by sequence comparison using programs such as BLAST run under appropriate conditions. In one aspect, the program is run under default parameters.

As understood by those of skill in the art, a “retro-inverso” refers to an isomer of a linear peptide in which the direction of the sequence is reversed (“retro”) and the chirality of each amino acid residue is inverted (“inverso”). Compared to the parent peptide, a helical retro-inverso peptide can substantially retain the original spatial conformation of the side chains but has reversed peptide bonds, resulting in a retro-inverso isomer with a topology that closely resembles the parent peptide, since all peptide backbone hydrogen bond interactions are involved in maintaining the helical structure. See Jameson et al., (1994) Nature 368:744-746 (1994) and Brady et al. (1994) Nature 368:692-693. The net result of combining D-enantiomers and reverse synthesis is that the positions of carbonyl and amino groups in each amide bond are exchanged, while the position of the side-chain groups at each alpha carbon is preserved. Unless specifically stated otherwise, it is presumed that any given L-amino acid sequence of the disclosure may be made into a D retro-inverso peptide by synthesizing a reverse of the sequence for the corresponding native L-amino acid sequence.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this disclosure that is a polynucleotide encompasses both the double-stranded form and each of two complementary single-stranded forms known or predicted to make up the double-stranded form.

A polynucleotide is composed of a specific sequence of four nucleotide bases; adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by =HIGH SCORE; Databases=non-redundant, GenBank+EMBL+DDBJ+PDB+GenBank CDS translations+SwissProtein+SPupdate+PIR. Details of these programs can be found at the following Internet address: http://www.ncbi.nlm.nih.gov/blast/Blast.cgi, last accessed on Nov. 26, 2007. Biologically equivalent polynucleotides are those having the specified percent homology and encoding a polypeptide having the same or similar biological activity.

The term “non-contiguous” refers to the presence of an intervening peptide, nucleotide, polypeptide or polynucleotide between a specified region and/or sequence. For example, two polypeptide sequences are non-contiguous because the two sequences are separated by a polypeptide sequences that is not homologous to either of the two sequences. Non-limiting intervening sequences are comprised of at least a single amino acid or nucleotide.

A “gene” refers to a polynucleotide containing at least one open reading frame (ORF) that is capable of encoding a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “express” refers to the production of a gene product such as RNA or a polypeptide or protein.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

Various proteins are also disclosed herein with their GenBank Accession Numbers for their human proteins and coding sequences. However, the proteins are not limited to human-derived proteins having the amino acid sequences represented by the disclosed GenBank Accession numbers, but may have an amino acid sequence derived from other animals, particularly, a warm-blooded animal (e.g., rat, guinea pig, mouse, chicken, rabbit, pig, sheep, cow, monkey, etc.).

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced there from.

Applicants have provided herein the polypeptide and/or polynucleotide sequences for use in gene and protein transfer and expression techniques described below. It should be understood, although not always explicitly stated that the sequences provided herein can be used to provide the expression product as well as substantially identical sequences that produce a protein that has the same biological properties. These “biologically equivalent” or “biologically active” polypeptides are encoded by equivalent polynucleotides as described herein. They may possess at least 60%., or alternatively, at least 65%, or alternatively, at least 70%, or alternatively, at least 75%, or alternatively, at least 80%, or alternatively at least 85%, or alternatively at least 90%, or alternatively at least 95% or alternatively at least 98%, identical primary amino acid sequence to the reference polypeptide when compared using sequence identity methods run under default conditions. Specific polypeptide sequences are provided as examples of particular embodiments. Modifications to the sequences to amino acids with alternate amino acids that have similar charge.

A “gene delivery vehicle” is defined as any molecule that can carry inserted polynucleotides into a host cell. Examples of gene delivery vehicles are liposomes, micelles, biocompatible polymers, including natural polymers and synthetic polymers; lipoproteins; polypeptides; polysaccharides; lipopolysaccharides; artificial viral envelopes; metal particles; and bacteria, or viruses, such as baculovirus, adenovirus and retrovirus, bacteriophage, cosmid, plasmid, fungal vectors and other recombination vehicles typically used in the art which have been described for expression in a variety of eukaryotic and prokaryotic hosts, and may be used for gene therapy as well as for simple protein expression.

Gene delivery vehicles also include DNA/liposome complexes, micelles and targeted viral protein-DNA complexes. Liposomes that also comprise a targeting antibody or fragment thereof can be used in the methods of this disclosure. To enhance delivery to a cell, the nucleic acid or proteins of this disclosure can be conjugated to antibodies or binding fragments thereof which bind cell surface antigens. In addition to the delivery of polynucleotides to a cell or cell population, direct introduction of the proteins described herein to the cell or cell population can be done by the non-limiting technique of protein transfection, alternatively culturing conditions that can enhance the expression and/or promote the activity of the proteins of this disclosure are other non-limiting techniques.

A polynucleotide of this disclosure can be delivered to a cell or tissue using a gene delivery vehicle. “Gene delivery,” “gene transfer,” “transducing,” and the like as used herein, are terms referring to the introduction of an exogenous polynucleotide (sometimes referred to as a “transgene”) into a host cell, irrespective of the method used for the introduction. Such methods include a variety of well-known techniques such as vector-mediated gene transfer (by, e.g., viral infection/transfection, or various other protein-based or lipid-based gene delivery complexes) as well as techniques facilitating the delivery of “naked” polynucleotides (such as electroporation, “gene gun” delivery and various other techniques used for the introduction of polynucleotides). The introduced polynucleotide may be stably or transiently maintained in the host cell. Stable maintenance typically requires that the introduced polynucleotide either contains an origin of replication compatible with the host cell or integrates into a replicon of the host cell such as an extrachromosomal replicon (e.g., a plasmid) or a nuclear or mitochondrial chromosome. A number of vectors are known to be capable of mediating transfer of genes to mammalian cells, as is known in the art and described herein.

As used herein, the term “detectable label” intends a directly or indirectly detectable compound or composition that is conjugated directly or indirectly to the composition to be detected, e.g., polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g. radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include, but are not limited to bioluminescence and chemiluminescence. Detectable luminescence response generally comprises a change in, or an occurrence of, a luminescence signal. Suitable methods and luminophores for luminescently labeling assay components are known in the art and described for example in Haugland, Richard P. (1996) Handbook of Fluorescent Probes and Research Chemicals (6^{th} ed.). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

In another aspect, the fluorescent label is functionalized to facilitate covalent attachment to a cellular component present in or on the surface of the cell or tissue such as a cell surface marker. Suitable functional groups, including, but not are limited to, isothiocyanate groups, amino groups, haloacetyl groups, maleimides, succinimidyl esters, and sulfonyl halides, all of which may be used to attach the fluorescent label to a second molecule. The choice of the functional group of the fluorescent label will depend on the site of attachment to either a linker, the agent, the marker, or the second labeling agent.

Attachment of the fluorescent label may be either directly to the cellular component or compound or alternatively, can by via a linker. Suitable binding pairs for use in indirectly linking the fluorescent label to the intermediate include, but are not limited to, antigens/antibodies, e.g., rhodamine/anti-rhodamine, biotin/avidin and biotin/strepavidin.

As used herein, the term “vector” refers to a nucleic acid construct deigned for transfer between different hosts, including but not limited to a plasmid, a virus, a cosmid, a phage, a BAC, a YAC, etc. A “viral vector” is defined as a recombinantly produced virus or viral particle that comprises a polynucleotide to be delivered into a host cell, either in vivo, ex vivo or in vitro. In some embodiments, plasmid vectors may be prepared from commercially available vectors. In other embodiments, viral vectors may be produced from baculoviruses, retroviruses, adenoviruses, AAVs, etc. according to techniques known in the art. In one embodiment, the viral vector is a lentiviral vector. Examples of viral vectors include retroviral vectors, adenovirus vectors, adeno-associated virus vectors, alphavirus vectors and the like. Further details as to modern methods of vectors for use in gene transfer may be found in, for example, Kotterman et al. (2015) Viral Vectors for Gene Therapy: Translational and Clinical Outlook Annual Review of Biomedical Engineering 17. Vectors that contain both a promoter and a cloning site into which a polynucleotide can be operatively linked are well known in the art. Such vectors are capable of transcribing RNA in vitro or in vivo and are commercially available from sources such as Agilent Technologies (Santa Clara, Calif.) and Promega Biotech (Madison, Wis.).

The term “promoter” as used herein refers to any sequence that regulates the expression of a coding sequence, such as a gene. Promoters may be constitutive, inducible, repressible, or tissue-specific, for example. A “promoter” is a control sequence that is a region of a polynucleotide sequence at which initiation and rate of transcription are controlled. It may contain genetic elements at which regulatory proteins and molecules may bind such as RNA polymerase and other transcription factors.

As used herein, the term “enhancer”, denotes sequence elements that augment, improve or ameliorate transcription of a nucleic acid sequence irrespective of its location and orientation in relation to the nucleic acid sequence to be expressed. An enhancer may enhance transcription from a single promoter or simultaneously from more than one promoter. As long as this functionality of improving transcription is retained or substantially retained (e.g., at least 70%, at least 80%, at least 90% or at least 95% of wild-type activity, that is, activity of a full-length sequence), any truncated, mutated or otherwise modified variants of a wild-type enhancer sequence are also within the above definition.

As used herein, the term “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell. The expression level of a gene may be determined by measuring the amount of mRNA or protein in a cell or tissue sample. In one aspect, the expression level of a gene from one sample may be directly compared to the expression level of that gene from a control or reference sample. In another aspect, the expression level of a gene from one sample may be directly compared to the expression level of that gene from the same sample following administration of a compound.

A “viral vector” is defined as a recombinantly produced virus or viral particle that comprises a polynucleotide to be delivered into a host cell, either in vivo, ex vivo or in vilro. Examples of viral vectors include retroviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827. In aspects where gene transfer is mediated by a retroviral vector, a vector construct refers to the polynucleotide comprising the retroviral genome or part thereof, and a therapeutic gene.

As used herein, “retroviral mediated gene transfer” or “retroviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. As used herein, retroviral vector refers to a viral particle capable of introducing exogenous nucleic acid into a cell through a viral or viral-like entry mechanism.

Retroviruses carry their genetic information in the form of RNA; however, once the virus infects a cell, the RNA is reverse-transcribed into the DNA form which integrates into the genomic DNA of the infected cell. The integrated DNA form is called a provirus.

In aspects where gene transfer is mediated by a DNA viral vector, such as an adenovirus (Ad) or adeno-associated virus (AAV), a vector construct refers to the polynucleotide comprising the viral genome or part thereof, and a transgene. Adenoviruses (Ads) are a relatively well characterized, homogenous group of viruses, including over 50 serotypes. See, e.g., International PCT Application No. WO 95/27071. Ads do not require integration into the host cell genome. Recombinant Ad derived vectors, particularly those that reduce the potential for recombination and generation of wild-type virus, have also been constructed. See, International PCT Application Nos. WO 95/00655 and WO 95/11984. Wild-type AAV has high infectivity and specificity integrating into the host cell's genome. See, Hermonat and Muzyczka (1984) Proc. Natl. Acad. Sci. USA 81:6466-6470 and Lebkowski et al. (1988) Mol. Cell. Biol. 8:3988-3996.

Vectors that contain both a promoter and a cloning site into which a polynucleotide can be operatively linked are well known in the art. Such vectors are capable of transcribing RNA in vitro or in vivo, and are commercially available from sources such as Stratagene (La Jolla, CA) and Promega Biotech (Madison, WI). In order to optimize expression and/or in vitro transcription, it may be necessary to remove, add or alter 5′ and/or 3′ untranslated portions of the clones to eliminate extra, potential inappropriate alternative translation initiation codons or other sequences that may interfere with or reduce expression, either at the level of transcription or translation. Alternatively, consensus ribosome binding sites can be inserted immediately 5′ of the start codon to enhance expression.

Cell penetrating peptides, (CPPs) or cell penetrating domains, as used herein, refer to short peptides that facilitate cellular uptake of various molecular cargos (from small chemical molecules to nanosize particles and large fragments of DNA). A “cargo”, such as a protein, is associated with the peptides either through chemical linkage via covalent bonds or through non-covalent interactions. The function of the CPPs are to deliver the cargo into cells, a process that commonly occurs through endocytosis with the cargo delivered to the endosomes of living mammalian cells. CPPs typically have an amino acid composition containing either a high relative abundance of positively charged amino acids such as lysine or arginine, or have sequences that contain an alternating pattern of polar/charged amino acids and non-polar, hydrophobic amino acids. It was previously reported that the human immunodeficiency virus transactivator of transcription (HIV-TAT) protein can be delivered to cells using a CPP or YGRKKRRQRRR (SEQ ID NO: 4).

A CPP employed in accordance with one aspect of the disclosure may include 3 to 35 amino acids, preferably 5 to 25 amino acids, more preferably 10 to 25 amino acids, or even more preferably 15 to 25 amino acids.

A CPP may also be chemically modified, such as prenylated near the C-terminus of the CPP. Prenylation is a post-translation modification resulting in the addition of a 15 (farneysyl) or 20 (geranylgeranyl) carbon isoprenoid chain on the peptide. A chemically modified CPP can be even shorter and still possess the cell penetrating property. Accordingly, a CPP, pursuant to another aspect of the disclosure, is a chemically modified CPP with 2 to 35 amino acids, preferably 5 to 25 amino acids, more preferably 10 to 25 amino acids, or even more preferably 15 to 25 amino acids.

A CPP suitable for carrying out one aspect of the disclosure may include at least one basic amino acid such as arginine, lysine and histidine. In another aspect, the CPP may include more, such as 2, 3, 4, 5, 6, 7, 8, 9, 10, or more such basic amino acids, or alternatively about 5%, 10%, 15%, 20%, 25%, 30%, 40%, 50% of the amino acids are basic amino acids. In one embodiment, the CPP contains at least two consecutive basic amino acids, or alternatively at least three, or at least five consecutive basic amino acids. In a particular aspect, the CPP includes at least two, three, four, or five consecutive arginine. In a further aspect, the CPP includes more arginine than lysine or histidine, or preferably includes more arginine than lysine and histidine combined.

CPPs may include acidic amino acids but the number of acidic amino acids should be smaller than the number of basic amino acids. In one embodiment, the CPP includes at most one acidic amino acid. In a preferred embodiment, the CPP does not include acidic amino acid. In a particular embodiment, a suitable CPP is the HIV-TAT peptide.

CPPs can be linked to a protein recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al. (2006) Bioconjugate Chem. 17:597-602).

CPPs also include cationic conjugates which also may be used to facilitate delivery of the proteins into the cells or tissue of interest. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids: more preferably 5 to 10 cationic amino acids or other cationic subunits.

Recombinant proteins anchoring CPP to the proteins can be generated to be used for delivery to cells or tissue.

The terms “culture” or “culturing” refer to the in vitro propagation of cells or organisms on or in media of various kinds. It is understood that the descendants of a cell grown in culture may not be completely identical (i.e., morphologically, genetically, or phenotypically) to the parent cell.

A “composition” is intended to mean a combination of active polypeptide, polynucleotide or antibody and another compound or composition, inert (e.g. a detectable label) or active (e.g. a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

“Administration” can be effected in one dose, continuously or intermittently throughout the course of treatment. Methods of determining the most effective means and dosage of administration are known to those of skill in the art and will vary with the composition used for therapy, the purpose of the therapy, the target cell being treated, and the subject being treated. Single or multiple administrations can be carried out with the dose level and pattern being selected by the treating physician. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, nasal administration, injection, and topical application.

An agent of the present disclosure can be administered for therapy by any suitable route of administration. It will also be appreciated that the preferred route will vary with the condition and age of the recipient, and the disease being treated.

The term “effective amount” refers to a quantity sufficient to achieve a desired effect. In the context of therapeutic or prophylactic applications, the effective amount will depend on the type and severity of the condition at issue and the characteristics of the individual subject, such as general health, age, sex, body weight, and tolerance to pharmaceutical compositions.

In the case of an in vitro application, in some embodiments the effective amount will depend on the size and nature of the application in question. It will also depend on the nature and sensitivity of the in vitro target and the methods in use. The skilled artisan will be able to determine the effective amount based on these and other considerations. The effective amount may comprise one or more administrations of a composition depending on the embodiment.

The agents and compositions for use in the methods of this disclosure can be concurrently or sequentially administered with effective or therapeutic agents. Non-limiting examples of administration include by one or more method comprising transdermally, urethrally, sublingually, rectally, vaginally, ocularly, subcutaneous, intramuscularly, intraperitoneally, intranasally, by inhalation or orally.

Thus, routes of administration applicable to the methods of the disclosure include intravenous, intranasal, intramuscular, urethrally, intratracheal, subcutaneous, intradermal, topical application, rectal, nasal, oral, inhalation, and other enteral and parenteral routes of administration. Routes of administration may be combined, if desired, or adjusted depending upon the agent and/or the desired effect. An active agent can be administered in a single dose or in multiple doses. Embodiments of these methods and routes suitable for delivery, include systemic or localized routes.

Parenteral routes of administration other than inhalation administration include, but are not limited to, topical, transdermal, subcutaneous, intramuscular, intraorbital, intracapsular, intraspinal, intrasternal, and intravenous routes, i.e., any route of administration other than through the alimentary canal. Parenteral administration can be conducted to effect systemic or local delivery of the inhibiting agent. Where systemic delivery is desired, administration typically involves invasive or systemically absorbed topical or mucosal administration of pharmaceutical preparations.

A “subject,” “individual” or “patient” is used interchangeably herein, and refers to a vertebrate, preferably a mammal, more preferably a human. Mammals include, but are not limited to, murines, rats, rabbits, simians, bovines, ovines, porcines, canines, felines, farm animals, sport animals, pets, equines, and primates, particularly humans.

“Cell,” “host cell” or “recombinant host cell” are terms used interchangeably herein and can be prokaryotic or eukaryotic. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

“Prokaryotic cells” that usually lack a nucleus or any other membrane-bound organelles and are divided into two domains, bacteria and archaea. In addition to chromosomal DNA, these cells can also contain genetic information in a circular loop called on episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium.

“Eukaryotic cells” comprise, or alternatively consist essentially of, or yet further consist of all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human,

“Treating,” “treatment,” or “ameliorating” of a disease includes: (1) preventing the disease, i.e., causing the clinical symptoms of the disease not to develop in a patient that may be predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. In one aspect, the term “treatment” excludes prophylaxis.

The term “suffering” as it related to the term “treatment” refers to a patient or individual who has been diagnosed with or is predisposed to a disease. A patient may also be referred to being “at risk of suffering” from a disease. This patient has not yet developed characteristic disease pathology, however are known to be predisposed to the disease due to family history, being genetically predispose to developing the disease, or diagnosed with a disease or disorder that predisposes them to developing the disease to be treated.

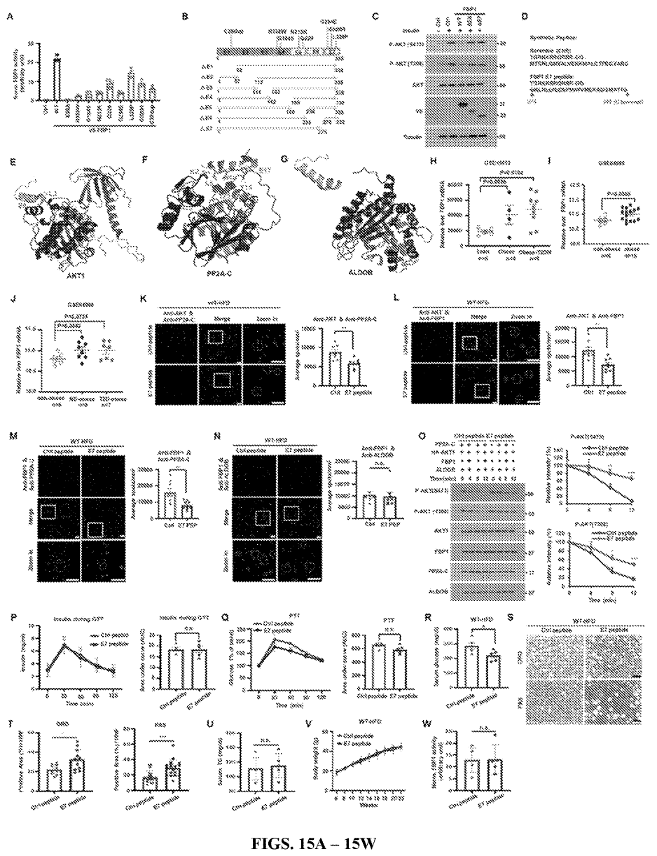

“FBP1” or “fructose (F) 1,6 bisphosphatase 1” is a gluconeogenesis regulatory enzyme, which catalyzes the hydrolysis of fructose 1,6-bisphosphate to fructose 6-phosphate and inorganic phosphate. “FBP1 E1-E7” or “FBP1 ΔE1-ΔE7”, e.g., “FBP1 E7” or “FBP1 ΔE7”, represents FBP1 deletion mutants, where each of E1-E7 (also referred to as ΔE1-ΔE7) lacks one FBP1 exon (E). Schematic of FBP1 exon deletion mutants is provided in FIG. 15B.

“Insulin resistance” is condition when cells in a subject's muscles, fat, and liver don't respond well to insulin and can't easily take up glucose from your blood. As a result, the pancreas makes more insulin to help glucose enter your cells. This can eventually lead to type 2 diabetes. Insulin resistance typically has no symptoms. For some patients, weight loss and exercise can help reverse insulin resistance.

### Descriptive Embodiments

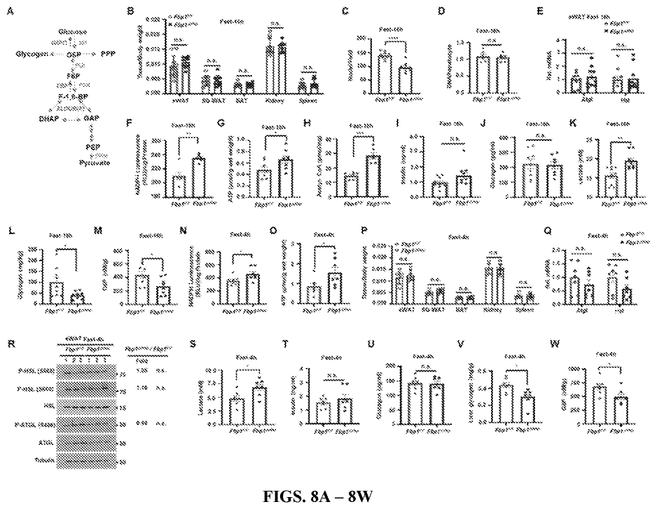

While not a direct target for insulin signaling, fructose (F) 1,6 bisphosphatase 1 (FBP1) is rate controlling for GNG, converting F1,6-P₂ to F6-P which is then converted to glucose (G) 6-P by phosphoglucoseisomerase (PGI) (6, 7). FBP1 deficiency is a rare inborn metabolic error (OMIM:229700), that causes severe hypoglycemia, lactic acidosis, seizures, hepatomegaly, hyperlipidemia, hepatosteatosis and liver damage in carbohydrate starved, but not fed, infants (8-10). While hypoglycemia and lactic acidosis are probably caused by low glycogen stores and abrogated GNG, there is no simple biochemical explanation for the hepatomegaly and elevated hepatic lipogenesis, which also affect non-starved older FBP1-deficient individuals (8). Applicant investigated hepatocyte specific Fbp1 knockout mice (Fbp1^{ΔHep}), initially generated for understanding FBP1's tumor suppressive function in hepatocellular carcinoma (HCC) (11) and found fasted Fbp1^{ΔHep} mice to be phenotypically and metabolically identical to starved FBP1-deficient infants. In addition to low glycogen stores and severe hypoglycemia, these mice manifest fasting-induced liver pathologies, including hepatomegaly, hepatosteatosis and hyperlipidemia. Unexpectedly and independently of its catalytic activity, Applicant discovered that FBP1 is a critical regulator of insulin signaling, serving as an endogenous “safety valve” that prevents insulin hyperresponsiveness and balances glucose and lipid metabolism. FBP1 negatively controls insulin signaling by nucleating a stable multiprotein complex that also contains Aldolase B (ALDOB), an enzyme that acts upstream to FBP1 in the GNG pathway (FIG. 8A), the catalytic subunit of protein phosphatase 2A (PP2A-C) which dephosphorylates AKT (12), and AKT, the key effector of insulin signaling (5). Complex formation which keeps AKT activation in check is enhanced by fasting and weakened during the fed state by insulin. Defective complex assembly causes hyperresponsiveness to the small amounts of insulin that are released in response to lipolysis-generated free fatty acids (FFA), thereby triggering hepatosteatosis and hyperlipidemia (13). Conversely, pharmacological complex disruption with an FBP1-derived peptide reversed diet-induced insulin resistance.

### Peptide, Polynucleotides, Vectors and Host Cells

Provided herein are isolated peptides comprising, consisting essentially of, or yet further consisting of a peptide identified herein as any one of FBP1 E1-E7, or an equivalent of each thereof.

In one aspect, the isolated peptide comprises, or consists essentially of or yet further comprises FBPIE7 or an equivalent of each thereof. In one aspect, the E7 peptide comprises, or consists essentially of, or consists of the peptide GKLRLLYECNPMAYVMEKAGGMATTG (SEQ ID NO. 1), or an equivalent thereof. The peptides and equivalents thereof can be detectably labeled.

Further provided are polynucleotides encoding the peptides, and the complements of these polynucleotides, that are optionally detectably labeled. The polynucleotide can be DNA or RNA. The polynucleotides can further comprise regulatory elements such as promoter or enhancers to facilitate expression or replication in cell-free systems or host cell systems. Thus, the disclosure provides vectors comprising the polynucleotides optionally operably linked to regulatory elements such as promoters and enhancers. Non-limiting examples of vectors include plasmids, viral vectors and non-viral vectors. Isolated host cells are further provided, wherein the cell comprises one or more of the polypeptide, polynucleotide, or vector. The host cells can be prokaryotic or eukaryotic cells. Further provided are compositions comprising a carrier and one or more of an isolated host cell, polypeptide, polynucleotide, or vector. The polypeptides, polynucleotides, vectors, host cells can be detectably labeled.

The polypeptide can further comprise a linker and/or a cell penetrating peptide on the amine or carboxy terminus

A “linker” or “peptide linker” refers to a peptide sequence linked to a polypeptide sequence at both ends of the linker peptide sequence. In one aspect, the linker is from about 1 to about 50 amino acid residues long or alternatively 1 to about 45, about 1 to about 40, about 1 to about 35, about 1 to about 30, about 1 to about 25, about 1 to about 20, about 1 to about 15, about 1 to about 10, about 1 to about 9, about 1 to about 8, about 1 to about 7, about 1 to about 6, about 1 to about 5, about 2 to about 40, about 2 to about 30, about 2 to about 25, about 2 to about 20, about 2 to about 15, about 2 to about 10, about 2 to about 9, about 2 to about 8, about 2 to about 7, about 2 to about 6, about 2 to about 5, about 3 to about 40, about 3 to about 30, about 3 to about 20, about 3 to about 15, about 3 to about 10, about 3 to about 9, about 3 to about 8, about 3 to about 7, about 3 to about 5, about 4 to about 40, about 4 to about 30, about 4 to about 20, about 4 to about 10, about 4 to about 8, about 4 to about 6, about 5 to about 40, about 5 to about 30, about 5 to about 20, about or 5 to about 10 amino acid residues long. In a particular aspect, the linker is from about 1 to about 20 amino acid residues long. In another particular aspect, the linker is from about 3 to 10 amino acid residues long. In some embodiments, the linker comprises, or consists essentially of, or consists of the peptide (GG)n wherein n is an integer from 1 to 10, optionally n is 1.

Thus, this disclosure also provides an isolated recombinant peptide comprising, or consisting essentially of, or yet further consisting of one or more of the FBP1 E1-E7 fragment or an equivalent of each thereof and a cell penetrating peptide.

The cell penetrating peptide (CPP) can be on the amine or the carboxy terminus, and in one aspect, comprises, consists essentially of, or yet further consisting of the TAT peptide (SEQ ID NO. 6) and an optional linker peptide between the E7 peptide and the CPP. In another aspect, the linker comprises, consists essentially of, or consists of YGRKKRRQRRR (SEQ ID NO: 4).

In one aspect, the E7 peptide comprises, or consists essentially of, or consists of the peptide GKLRLLYECNPMAYVMEKAGGMATTG (SEQ ID NO: 1), or an equivalent thereof or XGKLRLLYECNPMAYVMEKAGGMATTG, wherein X is no amino acid or an optional linker peptide (SEQ ID NO: 2). In one aspect, the recombinant peptide (e.g., the FBP1 recombinant peptide) comprises FBP1 E7 or an equivalent thereof and a cell penetrating peptide, that in one aspect, comprises, or consists essentially of, or consists of the peptide TAT (SEQ ID NO: 6) or YGRKKRRQRRR (SEQ ID NO: 4). Additional examples of cell penetrating peptides are provided herein, and known in the art.

In a further aspect, the E7 peptide comprises the peptide TAT. In some embodiments, the E7 peptide comprises, or consists essentially of, or yet further consists of the amino acid sequence TATXGKLRLLYECNPMAYVMEKAGGMATTG, wherein X is no amino acid or an optional linker peptide (SEQ ID NO: 2) or GKLRLLYECNPMAYVMEKAGGMATTGXTAT, wherein X is no amino acid or an optional linker peptide (SEQ ID NO: 3).

In some embodiments, the CPP comprises, or consists essentially of, or consists of the peptide YGRKKRRQRRR (SEQ ID NO: 4), or an equivalent thereof.

Thus, in accordance with any of the foregoing embodiments, a recombinant peptide (e.g., the FBP1 recombinant peptide) as described herein may comprise, or consist essentially of, or consist of the peptide YGRKKRRQRRRGGGKLRLLYECNPMAYVMEKAGGMATTG (SEQ ID NO: 5), or an equivalent thereof.

Also provided are polynucleotides encoding the FBP1 E1-E7 fragment or an equivalent thereof and an optional cell penetrating peptide and/or a linker peptide as described herein, or a complement of each thereof. The polynucleotide can be DNA or RNA.

Further provided are polynucleotides encoding the FBP1 E1 to E7 peptides or equivalents thereof, and the complements of these polynucleotides. In one aspect, the polynucleotide encodes the FBP1 E7 peptide or an equivalent thereof, and the complements of these polynucleotides.

The polynucleotide can be DNA or RNA. The polynucleotides can further comprise regulatory elements such as promoter or enhancers to facilitate expression or replication in vectors and/or host cell systems such as promoters and enhancers. Thus, the disclosure also provides vectors comprising the polynucleotides. Non-limiting examples are plasmids, viral vectors or non-viral vectors.

Isolated host cells are further provided, wherein the cell comprises one or more of the peptide, polypeptide, polynucleotide, and/or vector.

CPPs can be linked to the peptide or polypeptide recombinantly, covalently or non-covalently. A recombinant protein having a CPP peptide can be prepared in bacteria, such as E. coli, a mammalian cell such as a human HEK293 cell, or any cell suitable for protein expression. Covalent and non-covalent methods have also been developed to form CPP/protein complexes. A CPP, Pep-1, has been shown to form a protein complex and proven effective for delivery (Kameyama et al. (2006) Bioconjugate Chem. 17:597-602).

CPPs also include cationic conjugates which also may be used to facilitate delivery of the proteins into the cells or tissue of interest. Cationic conjugates may include a plurality of residues including amines, guanidines, amidines, N-containing heterocycles, or combinations thereof. In related embodiments, the cationic conjugate may comprise a plurality of reactive units selected from the group consisting of alpha-amino acids, beta-amino acids, gamma-amino acids, cationically functionalized monosaccharides, cationically functionalized ethylene glycols, ethylene imines, substituted ethylene imines, N-substituted spermine, N-substituted spermidine, and combinations thereof. The cationic conjugate also may be an oligomer including an oligopeptide, oligoamide, cationically functionalized oligoether, cationically functionalized oligosaccharide, oligoamine, oligoethyleneimine, and the like, as well as combinations thereof. The oligomers may be oligopeptides where amino acid residues of the oligopeptide are capable of forming positive charges. The oligopeptides may contain 5 to 25 amino acids; preferably 5 to 15 amino acids: more preferably 5 to 10 cationic amino acids or other cationic subunits.

Recombinant proteins anchoring CPP to the proteins can be generated to be used for delivery to cells or tissue.

In further embodiments, the peptides or polypeptides can be delivered to a cell, tissue or subject by administering an effective amount of a peptide and/or a polynucleotide encoding the peptide of the present disclosure, and compositions comprising the peptide or polynucleotide.

Peptides or polypeptides comprising the amino acid sequences for use in the methods of the disclosure can be prepared by expressing polynucleotides encoding the polypeptide or peptide sequences of this disclosure in an appropriate host cell. This can be accomplished by methods of recombinant DNA technology known to those skilled in the art. Accordingly, this disclosure also provides methods for recombinantly producing the polypeptides of this disclosure in a eukaryotic or prokaryotic host cells, as well as the isolated host cells used to produce the proteins. The proteins and polypeptides of this disclosure also can be obtained by chemical synthesis using a commercially available automated peptide synthesizer such as those manufactured by Perkin Elmer/Applied Biosystems, Inc., Model 430A or 431A, Foster City, CA, USA. The synthesized protein or polypeptide can be precipitated and further purified, for example by high performance liquid chromatography (HPLC). Accordingly, this disclosure also provides a process for chemically synthesizing the proteins of this disclosure by providing the sequence of the protein and reagents, such as amino acids and enzymes and linking together the amino acids in the proper orientation and linear sequence.

It is known to those skilled in the art that modifications can be made to any peptide to provide it with altered properties. Polypeptides of the disclosure can be modified to include unnatural amino acids. Thus, the peptides may comprise D-amino acids, a combination of D- and L-amino acids, and various “designer” amino acids (e.g., β-methyl amino acids, C-α-methyl amino acids, and N-α-methyl amino acids, etc.) to convey special properties to peptides. Additionally, by assigning specific amino acids at specific coupling steps, peptides with α-helices, β turns, β sheets, α-turns, and cyclic peptides can be generated. Generally, it is believed that α-helical secondary structure or random secondary structure is preferred.

In a further embodiment, subunits of polypeptides that confer useful chemical and structural properties can be use or will be chosen. For example, peptides comprising D-amino acids may be resistant to L-amino acid-specific proteases in vivo. Modified compounds with D-amino acids may be synthesized with the amino acids aligned in reverse order to produce the peptides of the disclosure as retro-inverso peptides. In addition, the present disclosure envisions preparing peptides that have better defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂NH—R₂, where R₁, and R₂ are amino acid residues or sequences. A reduced peptide bond may be introduced as a dipeptide subunit. Such a molecule would be resistant to peptide bond hydrolysis, e.g., protease activity. Such molecules would provide ligands with unique function and activity, such as extended half-lives in vivo due to resistance to metabolic breakdown, or protease activity. Furthermore, it is well known that in certain systems constrained peptides show enhanced functional activity (Hruby (1982) Life Sciences 31:189-199 and Hruby et al. (1990) Biochem J. 268:249-262); the present disclosure provides a method to produce a constrained peptide that incorporates random sequences at all other positions.

Non-classical amino acids may be incorporated in the peptides of the disclosure in order to introduce particular conformational motifs, examples of which include without limitation: 1,2,3,4-tetrahydroisoquinoline-3-carboxylate (Kazrnierski et al. (1991) J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski & Hruby (1991) Tetrahedron Lett. 32(41):5769-5772); 2-aminotetrahydronaphthalene-2-carboxylic acid (Landis (1989) Ph.D. Thesis, University of Arizona); hydroxy-1,2,3,4-tetrahydroisoquinoline-3-carboxylate (Miyake et al. (1989) J. Takeda Res. Labs. 43:53-76) histidine isoquinoline carboxylic acid (Zechel et al. (1991) Int. J. Pep. Protein Res. 38(2):131-138); and HIC (histidine cyclic urea), (Dharanipragada et al. (1993) Int. J. Pep. Protein Res. 42(1):68-77) and (Dharanipragada et al. (1992) Acta. Crystallogr. C. 48:1239-1241).

The following amino acid analogs and peptidomimetics may be incorporated into a peptide to induce or favor specific secondary structures: LL-Acp (LL-3-amino-2-propenidone-6-carboxylic acid), a β-turn inducing dipeptide analog (Kemp et al. (1985) J. Org. Chem. 50:5834-5838); β-sheet inducing analogs (Kemp et al. (1988) Tetrahedron Lett. 29:5081-5082); β-turn inducing analogs (Kemp et al. (1988) Tetrahedron Lett. 29:5057-5060); α-helix inducing analogs (Kemp et al. (1988) Tetrahedron Lett. 29:4935-4938); α-turn inducing analogs (Kemp et al. (1989) J. Org. Chem. 54:109:115); analogs provided by the following references: Nagai & Sato (1985) Tetrahedron Lett. 26:647-650; and DiMaio et al. (1989) J. Chem. Soc. Perkin Trans. p. 1687; a Gly-Ala turn analog (Kahn et al. (1989) Tetrahedron Lett. 30:2317); amide bond isostere (Clones et al. (1988) Tetrahedron Lett. 29:3853-3856); tetrazole (Zabrocki et al. (1988) J. Am. Chem. Soc. 110:5875-5880); DTC (Samanen et al. (1990) Int. J. Protein Pep. Res. 35:501:509); and analogs taught in Olson et al. (1990) J. Am. Chem. Sci. 112:323-333 and Garvey et al. (1990) J. Org. Chem. 56:436. Conformationally restricted mimetics of beta turns and beta bulges, and peptides containing them, are described in U.S. Pat. No. 5,440,013.

It is known to those skilled in the art that modifications can be made to any peptide by substituting one or more amino acids with one or more functionally equivalent amino acids that does not alter the biological function of the peptide. In one aspect, the amino acid that is substituted by an amino acid that possesses similar intrinsic properties including, but not limited to, hydrophobicity, size, or charge. Methods used to determine the appropriate amino acid to be substituted and for which amino acid are known to one of skill in the art. Non-limiting examples include empirical substitution models as described by Dayhoff et al. (1978) In Atlas of Protein Sequence and Structure Vol. 5 suppl. 2 (ed. M. O. Dayhoff), pp. 345-352. National Biomedical Research Foundation, Washington DC; PAM matrices including Dayhoff matrices (Dayhoff et al. (1978), supra, or JTT matrices as described by Jones et al. (1992) Comput. Appl. Biosci. 8:275-282 and Gonnet et al. (1992) Science 256:1443-1145; the empirical model described by Adach & Hasegawa (1996) J. Mol. Evol. 42:459-468; the block substitution matrices (BLOSUM) as described by Henikoff & Henikoff (1992) Proc. Natl. Acad. Sci. USA 89:1-1; Poisson models as described by Nei (1987) Molecular Evolutionary Genetics. Columbia University Press, New York.; and the Maximum Likelihood (ML) Method as described by Müller et al. (2002) Mol. Biol. Evol. 19:8-13.

In one aspect, the peptides and/or polynucleotides of this disclosure are detectably labeled.

### Compositions

Further provided are compositions comprising a carrier and one or more of an isolated host cell, peptide, polypeptide, polynucleotide, and/or vector. The peptides, polypeptides, polynucleotides, vectors, host cells can be detectably labeled.

In another aspect, any of the above compositions further comprises a carrier. The carrier can be a solid phase carrier, a gel, an aqueous liquid carrier, a paste, a liposome, a micelle, albumin, polyethylene glycol, a pharmaceutically acceptable polymer, or a pharmaceutically acceptable carrier, such a phosphate buffered saline.

The compositions of the disclosure can be manufactured by methods well known in the art such as conventional granulating, mixing, dissolving, encapsulating, lyophilizing, or emulsifying processes, among others. Compositions may be produced in various forms, including granules, precipitates, or particulates, powders, including freeze dried, rotary dried or spray dried powders, amorphous powders, injections, emulsions, elixirs, suspensions or solutions. Compositions may optionally contain stabilizers, pH modifiers, surfactants, bioavailability modifiers and combinations of these.

Compositions may be prepared as liquid suspensions or solutions using a sterile liquid, such as oil, water, alcohol, and combinations thereof. Pharmaceutically suitable surfactants, suspending agents or emulsifying agents, may be added for oral or parenteral administration. Suspensions may include oils, such as peanut oil, sesame oil, cottonseed oil, corn oil and olive oil. Suspension preparation may also contain esters of fatty acids, such as ethyl oleate, isopropyl myristate, fatty acid glycerides and acetylated fatty acid glycerides. Suspension compositions may include alcohols, such as ethanol, isopropyl alcohol, hexadecyl alcohol, glycerol and propylene glycol. Ethers, such as poly(ethyleneglycol), petroleum hydrocarbons, such as mineral oil and petrolatum, and water may also be used in suspension compositions.

The compositions of this disclosure are formulated for pharmaceutical administration to a mammal, preferably a human being. Such compositions of the disclosure may be administered in a variety of ways, preferably topically or by injection.

Sterile injectable forms of the compositions of this disclosure may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation. Compounds may be formulated for parenteral administration by injection such as by bolus injection or continuous infusion. A unit dosage form for injection may be in ampoules or in multi-dose containers.

In addition to dosage forms described above, pharmaceutically acceptable excipients and carriers and dosage forms are generally known to those skilled in the art and are included in the disclosure. It should be understood that a specific dosage and treatment regimen for any particular subject will depend upon a variety of factors, including the activity of the specific antidote employed, the age, body weight, general health, sex and diet, renal and hepatic function of the subject, and the time of administration, rate of excretion, drug combination, judgment of the treating physician or veterinarian and severity of the particular disease being treated.

### Polypeptide Conjugates

Also provided are methods comprising delivering or administering an effective amount of a peptide conjugate comprising, or alternatively consisting essentially of, or alternatively consisting of, a carrier covalently or non-covalently linked to an isolated polypeptide of the disclosure. In some embodiments, the carrier comprises a liposome, or alternatively a micelle, or alternatively a pharmaceutically acceptable polymer, or a pharmaceutically acceptable carrier.

### Methods

In one aspect, this disclosure provides a method to deliver an FBP1 E7 peptide to a cell or tissue by contacting the cell or tissue with a peptide, polynucleotide, vector or host cell of this disclosure. The FBP1 E7 polypeptide can further comprise a CPP on the amine and/or carboxy terminus, with an optional linker peptide between the E7 polypeptide and the CPP.

The contacting can be in vitro or in vivo.

In one aspect, the present disclosure provides a method to disrupt the interaction of AKT and/or PP2A-C and disrupt their interactions with FBP1 by contacting a composition comprising AKT and/or PP2A, and FBP1 with an effective amount of an FBP1 E7 fragment or an equivalent thereof. The FBP1 E7 polypeptide can further comprise a CPP on the amine and/or carboxy terminus, with an optional linker peptide between the E7 polypeptide and the CPP.

The contacting is in vitro or in vivo.

Further provided is a method for one or more of: activating AKT, sensitizing insulin, or reversing obesity-induced glucose intolerance in a subject in need thereof, comprising administering to the subject an effective amount of an FBP1 E7 polypeptide or an equivalent thereof or polynucleotide or vector as described herein. The FBP1 E7 polypeptide can further comprise a CPP on the amine and/or carboxy terminus, with an optional linker peptide between the E7 polypeptide and the CPP. In one aspect the subject being treated exhibits one or more of glucose intolerance, insulin resistance, diabetes, or obesity-induced glucose intolerance.

Further provided herein is a method for treating or preventing hypoglycemia, hepatosteatosis, hepatomegaly, hepatosteatosis, and/or hyperlipidemia, in a subject in need thereof, comprising administering to the subject an effective amount of an FBP1 E7 polypeptide or an equivalent thereof or polynucleotide or vector as described herein. The FBP1 E7 polypeptide can further comprise a CPP on the amine and/or carboxy terminus, with an optional linker peptide between the FBP1 E7 polypeptide and the CPP. In one aspect, the subject is suffering from or predisposed to suffering from one or more of preventing hypoglycemia, hepatosteatosis, hepatomegaly, hepatosteatosis, and/or hyperlipidemia.

The subject to be treated can be a mammal such as a human patient.

Route of administration for the methods can be any methods disclosed herein, including but not limited to injection, intraperitoneally, parenteral administration, inhalation, or topical application.

The compositions administered in accordance with the disclosure can be packaged in dosage unit form for ease of administration and uniformity of dosage. The term “unit dose” or “dosage” refers to physically discrete units suitable for use in a subject, each unit containing a predetermined quantity of the composition calculated to produce the desired responses in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described herein.

The peptides, polynucleotides, vectors or compositions of the present disclosure can be administered by parenteral (e.g., intramuscular, intraperitoneal, intravenous, ICV, intracisternal injection or infusion, subcutaneous injection, or implant), oral, by inhalation spray nasal, vaginal, rectal, sublingual, urethral (e.g., urethral suppository) or topical routes of administration (e.g., gel, ointment, cream, aerosol, etc.) and can be formulated in suitable dosage unit formulations containing conventional non-toxic pharmaceutically acceptable carriers, adjuvants, excipients, and vehicles appropriate for each route of administration.

In some embodiments, any of the peptides, polynucleotides, vectors or compositions disclosed herein, are administered to the subject at least 1, 2, 3, 4, 5, 6, 7, 8, 9, or 10 times a day. In some embodiments, any of conjugates or compositions disclosed herein are administered to the subject at least 1, 2, 3, 4, 5, 6, 7, 8, 9, 10, 11, 12, 13, 14, 15, 16, 17, 18, 19, or 20 times a week. In some embodiments, any of the peptides, polynucleotides, vectors or compositions disclosed herein, are administered to the subject at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 times a month. In some embodiments, any of the peptides, polynucleotides, vectors or compositions disclosed herein, are administered to the subject at least every 1, 2, 3, 4, 5, 6, 7, 8, 9, or 10 days. In some embodiments, any of the peptides, polynucleotides, vectors or compositions disclosed herein, are administered to the subject at least every 1, 2, 3, 4, 5, 6, 7, 8, 9, 10, 11, 12, 13, or 14 weeks. In some embodiments, any of the peptides, polynucleotides, vectors or compositions disclosed herein are administered to the subject for a period of at least 1, 2, 3, 4, 5, 6, 7, 8, 9, or 10 days. In some embodiments, any of the peptides, polynucleotides, vectors or compositions disclosed herein are administered to the subject for a period of at least 1, 2, 3, 4, 5, 6, 7, 8, 9, 10, 11, 12, 13, 14, 15, 16, 17, 18, 19, or 20 weeks. In some embodiments, any of the conjugates or compositions disclosed herein are administered to the subject for a period of at least 1, 2, 3, 4, 5, 6, 7, 8, 9, 10, 12, 13, 14, 15, 16, 17, 18, 19, or 20 months.

In one aspect, the methods or compositions further comprise administration of an additional therapeutic agent. The methods can be combined with other suitable therapy.

Examples of the above methods are provided herein.

### Kits

An aspect of the disclosure provides a kit comprising a polypeptide, polynucleotide, cell or vector as described herein and instructions to use.

Kits may further comprise suitable packaging and/or instructions for use of the compositions. The compositions can be in a dry or lyophilized form, in a solution, particularly a sterile solution, or in a gel or cream. The kit may contain a device for administration or for dispensing the compositions, including, but not limited to, syringe, pipette, transdermal patch and/or microneedle.

The kits may include other therapeutic compounds for use in conjunction with the compounds described herein. These compounds can be provided in a separate form or mixed with the compounds of the present disclosure.

The kits will include appropriate instructions for preparation and administration of the composition, side effects of the compositions, and any other relevant information. The instructions can be in any suitable format, including, but not limited to, printed matter, videotape, computer readable disk, or optical disc.

In another aspect of the disclosure, kits for treating a subject who suffers from or is susceptible to the conditions described herein are provided, comprising a container comprising a dosage amount of a composition as disclosed herein, and instructions for use. The container can be any of those known in the art and appropriate for storage and delivery.

Kits may also be provided that contain sufficient dosages of the effective composition or compound to provide effective treatment for a subject for an extended period, such as a week, 2 weeks, 3, weeks, 4 weeks, 6 weeks, or 8 weeks or more.

### EXPERIMENTAL

### Materials and Methods

### Mice

Fbp1^{F/F} mice were generated by Dr. M. Celeste Simon (University of Pennsylvania, Philadelphia) (11) and crossed to Alb-Cre mice to generate Fbp1^{ΔHep} mice at UCSD. All mice were backcrossed into the BL6 background at least nine generations, and only male mice were used in most experiments. Mice were maintained in filter-topped cages on autoclaved food and water with a 12 h light (6 am-6 pm)/dark (6 pm-6 am) cycle. Experiments were performed according to UCSD Institutional Animal Care and Use Committee and NIH guidelines and regulations. Dr. Karin's Animal protocol S00218 was approved by the UCSD Institutional Animal Care and Use Committee. Where indicated, mice were fed with HFD (#S3282, Bio-serv) for a total of 12 wk, starting at 7 wk-of-age. Body weight and food consumption were monitored bi-weekly throughout the entire feeding period (32). Mice were starved for 4 h or 16 h before blood collection and sacrifice and liver and adipose tissue were excised and weighed. Where indicated, male Fbp1^{F/F} and Fbp1^{ΔHep} mice (8 wo) were intraperitoneally (i.p.) injected with MK2206 (100 mg/kg) or Torin1 (10 mg/kg) on 2 sequential days prior to fasting from 6 μm to 10 am, at which point the mice were euthanized for blood and tissue collection. The number of mice per experiment and their ages are indicated in the figure legends.

For streptozotocin treatment, 8-10 wo Fbp1^{F/F} and Fbp1^{ΔHep} male and female mice were i.p. injected with 150 mg/kg STZ. Mice were monitored daily for food and water consumption. Blood glucose was measured daily at random. After 5-7 days, mice with substantial hyperglycemia were fasted for 4 h before euthanasia and liver and blood analyses.

### Cell Culture and Reagents

Human HCC HepG2 and Huh7 cell lines were purchased from ATCC (HB-8065) and Cell bank, respectively. Huh7, HepG2, Caco-2 and 293T cells were tested regularly to confirm they are mycoplasma negative, and cultured in low glucose DMEM (Life technologies, 11885084), MEM (Life technologies, 11095080, and DMEM (Life technologies, 2366044), respectively, plus 10% fetal bovine serum (FBS) (Gibco), penicillin (100 mg/ml) and streptomycin (100 mg/ml). The HK-2 kidney cell line was provided by Dr. Celeste Simon and cultured in Keratinocyte-SFM medium supplemented with human recombinant epidermal growth factor (EGF) and bovine pituitary extract (BPE) (Life Technologies) (6). Cells were incubated at 37 °C in a humidified chamber with 5% CO2. Cells were treated with insulin (Sigma 11061-68-0) at the times and concentrations indicated in figure legends.

### Human Hepatocytes

Donor livers rejected for transplantation were obtained via Lifesharing OPO as a part of T. Kisseleva's research program. Donors, who had no history of alcohol abuse, body-mass index 33.82, had no liver fibrosis and minimal liver steatosis, was qualified in this study conducted under IRB171883XX (approved on November 2017 by UCSD human Research Protection Program, under the title “Unused liver from deceased donors: role of myofibroblasts in liver fibrosis”) (33). For hepatocytes isolation, the livers were placed on top of an ice pan covered by a plastic bag and then a sterile hood. Catheters were inserted into the major portal and/or hepatic vessels, and the tissue was perfused with cold organ-preservation solution or EMEM (Corning Cellgro cat no. 15-010-CM)+25 mM HEPES (Corning Cellgro cat. no. 25-060-CI). The catheters were then secured into the vessels either by sutures or surgical-grade glue. The liver tissue was then placed in a sterile plastic bag and connected to a peristaltic pump, and the tissue was perfused with HBSS (without calcium and magnesium or phenol red) (Hyclone cat no. SH30588.02) supplemented with 1.0 mM EGTA without recirculation for 10-20 min. Finally, the liver specimen was perfused with DMEM containing 0.1 mg ml⁻¹ of collagenase (VitaCyte cat no. 001-2030) and 0.02 mg ml⁻¹ of protease (VitaCyte cat. no. 003-1000). Perfusion was stopped when the liver tissue began to show fissures and separation from the liver capsule. The liver tissue was then removed from the plastic bag and placed in a sterile plastic beaker that contained warmed (37 °C) DMEM supplemented with 5% FBS, 1% sodium pyruvate and 1% antibiotic. The cell suspension was filtered through sterile nylon-mesh-covered funnels to remove cellular debris and clumps of undigested tissue. Hepatocytes were isolated by low-speed centrifugation at 80 g for 5 min at 18 °C. The hepatocyte pellets were gently resuspended in a Percoll (Sigma cat. no. GE17-0891-09) gradient and centrifuged at 100 g for 10 minutes at 18 °C. The supernatant was removed by aspiration, and the pellets were saved and resuspended in warmed (37 °C) DMEM and the centrifugation step was repeated. The hepatocytes were plated in collagen coated 6-wells and after attached, were incubated with shCtrl and shFBP1 lentivirus for 24 h. After recovery for 24-48 h, the cells were stimulated with insulin for the indicated times and concentrations after 6 h of serum starvation.

### Constructs

V5-FBP1, V5-FBP1^{E98A} and FBP1 truncation mutations (ΔE1-ΔE7) were generated in Dr. M. Celeste Simon's lab. Flag-ALDOB, Flag-PP2A-C, HA-AKT1 were kindly provided by Drs. Huiyong Yin (Chinese Academy of Sciences, Shanghai, China) and Alexandra C. Newton (University of California at San Diego, USA), respectively. FBP1 (TRCN0000050034, TRCN0000050035) and PP2A-C(TRCN0000002483, TRCN0000002486) shRNAs were purchased from Sigma. SgALDOB was constructed by cloning the guide sequences into the BsmBI site of the lentiCRISPR v2-puro vector. Human FBP1 mutants were constructed by Q5 Site-Directed Mutagenesis Kit (NEB, E0554S) in the PCDH-V5-FBP1 vectors. Primers are listed in Tables 1-2.

### Plasmid Transfection and Virus Infection

Expression vectors were transfected into 293T cells or HCC cell lines using Lipofectamine 3000 (Invitrogen, L300008) following the manufacturer's protocol. Lentiviruses were produced by co-transfecting PSPAX2 (4 μg), PMD2. G (4 μg) and PCDH-V5-FBP1 (8 μg) or FBP1 mutants (8 μg), PLKO.1 (8 μg) or lentiCRISPERv2 (8 μg) vectors. After 8 h medium was changed and fresh DMEM plus 10% FBS was added. Virus containing media were harvested 48-64 h later and filtered by 0.45 μm Steriflip filter (Millipore). HepG2 and Huh7 cells were infected with 1 ml virus containing medium with 8 μg/ml polybrene for 24 h. Cells were allowed to recover in complete medium for 24-48 h and then selected with puromycin. After 48 h surviving cell pools were used in the different experiments.

### Immunoblot Analysis and Nuclear Extraction

Cells were harvested and lysed in RIPA buffer (50 mM Tris-HCl, pH 7.4, 150 mM NaCl, 1% Triton X-100, 1% sodium deoxycholate, 0.1% SDS, 1 mM EDTA) supplemented with complete protease inhibitor cocktail (34). Livers were homogenized in a Dounce homogenizer (Thomas Scientific, NJ) with 30 strokes in RIPA buffer with complete protease inhibitor cocktail. The proteins separated by SDS-PAGE and transferred to polyvinylidene difluoride (PVDF) membranes, blocked in 5% nonfat milk, and incubated with the indicated primary antibodies overnight. Second antibodies were added for another 1 h and detected with Clarity Western ECL Substrate (Biorad). Immunoreactive bands were exposed in an automatic X-ray film processor. Nuclear extraction was performed with the NE-PER™ Nuclear and Cytoplasmic Extraction Reagent kit (Thermo Fisher, 78833), following manufacturer's instructions. After extraction, nuclear and cytoplasmic extracts were separated by SDS-PAGE and analyzed by immunoblotting as above.

### Histology

Livers were dissected, fixed in 4% paraformaldehyde and embedded in paraffin. 5 μm thick sections were stained with hematoxylin and eosin (H&E) (Leica, 3801615, 3801571). For frozen block preparation, livers were embedded in Tissue-Tek OCT compound (Sakura Finetek), sectioned, and stained with Oil Red O to visualize TG accumulation. Liver PAS staining was performed with PAS kit (Thermo Fisher Scientific, #87007), using the manufacture's protocol.

### Measurements of Metabolites and Hormones

Liver TG, serum TG and serum cholesterol were measured with Triglyceride Colorimetric Assay Kit (Cayman Chemical #10010303) and Cholesterol Fluorometric Assay Kit (Cayman Chemical #10007640), respectively, according to manufacturer protocols. Serum insulin and glucagon concentrations were determined by Mouse Insulin ELISA KIT (Invitrogen, EMMINS) and Glucagon Quantikine ELISA Kit (R&D Systems, DGCGO), respectively, following manufacturer protocols. Liver NADPH and NADP were measured by NADP/NADPH-Glo™ Assays (Promega #G9081) according to manufacturer's protocol. ALT and AST assays were performed with ALT(GPT) Reagent (Thermo Scientific™, TR71121) and AST/GOT Reagent (Thermo ScientificTm, TR70121), respectively, according to the manufacturer's protocol. ATP, Acetyl-CoA and G6P concentrations were determined by ATP assay kit (Abcam, ab83355), Acetyl-Coenzyme A Assay Kit (Sigma, MAK039) and PicoProbe™ Glucose-6-Phosphate Fluorometric Assay Kit (Biovision, K687), respectively according to the manufacturers protocols. Glucose and lactate were measured with Glucose colorimetric assay kit (Cayman Chemical #10009582) and Lactate Colorimetric/Fluorometric Assay Kit (Biovision, K607), respectively, according to the manufacturer's protocols.

[U-¹³C]Glucose, ¹³C-Lactate and D₂O Labeling

D-glucose (U-¹³C6, 99%) and ¹³C-lactate were purchased from Cambridge Isotope Laboratories (NC9337143, CLM-1579). 8 wo Fbp1^{F/F} and Fbp1^{ΔHep} mice were fasted overnight from 6 pm-10 am and 45 min before sacking were injected intravenously (i.v.) with 500 mg/kg [U-¹³C]glucose as previously described (35, 36). For lactate labeling, mice were i.v. injected with 0.5 mg/g sodium L-lactate (¹³C3) for 3 times 1 h before sacking (37). Liver and serum were collected for metabolomic analysis. To measure DNL, 8 wo Fbp1^{F/F} and F-bp1^{ΔHep} mice were i.p. injected with 0.035 ml/body weight D₂O (Sigma) in 0.9% NaCl, and drinking water was replaced with 8% enriched D₂O (38). Mice were given D₂O for 22-24h and starved for 16 h before sacrifice, plasma and liver were collected and immediately snap-frozen in liquid nitrogen.

### Stable-Isotope Tracer Metabolomics

Sample extraction methods: Frozen liver samples of Fbp1^{F/F} and Fbp1^{ΔHeP} (~40 mg) were transferred to 2-mL tubes containing 2.8 mm ceramic beads (Omni International) and 0.45 ml ice-cold 50% methanol/20 μM L-norvaline was added. Tubes were shaken (setting 5.5) for 30 s on a Bead Ruptor 12 (Omni International), quickly placed on ice, and frozen at −80 °C overnight. Thawed samples were centrifuged at 15,000×g for 10 minutes at 4 °C. The supernatant was then transferred to a new tube, mixed with 0.225 ml chloroform, and centrifuged at 10,000×g for 10 minutes at 4 °C. This produced a two-phase separation. Portions (100 μl) of the top phase were dried (Speedvac) for analyses of polar metabolites. The lower phase was dried (Speedvac) for analysis of fatty acids. Plasma samples (5 or 10 μl) were mixed with 4 volumes of acetone (pre-cooled to −20 °C), left at −20 °C for 1 h, and centrifuged at 14,000×g for 10 minutes at 4 °C. Supernatants were dried by Speedvac.

Metabolite derivatization and GC-MS run conditions: Polar metabolites except for glucose and sugar-phosphates were derivatized using isobutylhydroxylamine and MTBSTFA, and analyzed by GC-MS as described (39). Plasma samples for glucose analysis were derivatized first with 30 μl ethylhydroxylamine (Sigma) at 20 mg/ml in pyridine for 20 min at 80 °C, and secondarily with 30 μl BSTFA (Thermo) for 60 min at 80 °C. Samples were transferred to autosampler vials with inserts and analyzed using an Rxi-5 ms column (15 m×0.25 i.d.×0.25 μm, Restek) installed in a Shimadzu QP-2010 Plus gas chromatograph-mass spectrometer (GC-MS). The GC-MS was programmed with an injection temperature of 250 °C, 1.0 μl injection volume and split ratio 1/10. The GC oven temperature was initially 130 °C for 4 min, rising to 230 °C at 6 °C/min, and to 280 °C at 60 °C/min with a final hold at this temperature for 2 min. GC flow rate, with helium as the carrier gas, was 50 cm/s. The GC-MS interface temperature was 300 °C and (electron impact) ion source temperature was 200 °C, with 70 eV ionization voltage. The main glucose peak eluted at 13.7 min, and fragments of m/z 319 (contains 4 glucose carbons; C₁₃H₃₁O₃Si₃) and m/z 205 (contains 2 glucose carbons; overall formula C₈H₂₁O₂Si₂) were used to analyze ¹³C-glucose labeling as described (39). Liver samples labeled with ¹³C-lactate for sugar-phosphate analysis were derivatized first with 30 μl pentafluorobenzyl-hydroxylamine (Alfa Aesar) 20 mg/ml in pyridine for 60 min at 37 °C, and secondarily with 30 μl BSTFA (Thermo) for 60 min at 37 °C. The GC-MS method was as described above, except that injection volume was 2 μl, and initial GC oven temperature was 200 °C for 4 min, rising to 280 °C at 8 °C/min, with a final hold at this temperature for 2 min. F6-P eluted as a pair of peaks at 10.33 and 10.49 min. The earlier peak was analyzed for ¹³C-labeling (fragment of m/z 459 containing 3 glucose carbons; C₁₅H₄₀O₆Si₄P). Fatty acids (palmitate and stearate) were derivatized and analyzed for labeling as described (the methyl stearate ion m/z 298 was analyzed) (39).

### Size Exclusion Chromatography

Liver samples were fractionated on a Superdex 200 HR 10/30 column (Pharmacia Biotech, Cat #17-1088-01) using an AKTA FPLC system (GE). Column was equilibrated with and samples were fractionated in CHAPS lysis buffer (25 mM HEPES pH 7.4, 150 mM NaCl, 1 mM EDTA, and 0.3% CHAPS). 0.5 mL of sample at 10 mg/mL (5 mg protein total) in CHAPS lysis buffer was injected onto the column and fractionated at a 0.5 mL/min flow rate over 1.2 column volumes. 0.25 mL fractions were collected. The injection loop was thoroughly rinsed, and blank runs were performed between each sample.

Molecular weights ranges within fractions were approximated through comparison with a standard curve (Kav of protein standards v. log(MW)). Kav of Gel Filtration Protein Standards (Bio-Rad, Cat #1511901) of known molecular weight were determined under identical fractionation conditions as described for samples and calculated using the following equation where Ve is elution volume, Vo is void volume, and Vt is the total volume of the column:

$${K}_{av} = \frac{Ve - Vo}{Vt - Vo}$$

Elution volumes were determined by monitoring inline absorbance at 280 nm as the standards eluted. Peak elution volumes were determined using Unicorn 5.1 Software (GE). Void volume was similarly determined by monitoring elution of 1 mg of blue dextran (Sigma D4772). Total volume of the column was provided by the manufacturer.

### FBP1 Activity Assay

FBP1 activity was measured with Fructose-1,6-Bisphosphatase Activity Assay Kit (Colorimetric) (Abcam, ab273329). Briefly, 20 μg lysates of control Huh7 cells, or Huh7 cells expressing WT and mutant forms of FBP1 were prepared in 500 μl ice-cold FBP assay buffer. (NH₄)₂SO₄ was used to precipitate the proteins and remove small molecules that could interfere with the assay. The precipitated proteins were spun down and resuspend in FBP assay buffer. The samples were incubated with the reaction mix containing FBP converter, FBP probe, FBP developer and FBP substrate and absorbance at OD=450 was measured in a kinetic mode for 5-60 min at 37 °C. FBP1 activity was normalized according to its expression level determined by immunoblotting.

### GTT, PTT, and ITT

For GTT and PTT, Fbp1^{F/F} and Fbp1^{ΔHep} mice that were HFD fed for 12 wk or NCD-8 wo mice were fasted for 12-14 h and then given 1 g/kg glucose or sodium pyruvate (2 g/kg, Sigma) by i.p. injection. Blood glucose was measured before injection and every 30 min thereafter with a glucometer (OneTouch Ultra 2, One Touch) on blood from superficial tail incision. 8 wo Fbp1^{F/F} and Fbp1^{ΔHep} mice or 18 wo Fbp1^{F/F} and Fbp1^{ΔHep} mice that were HFD fed for 12 wk, fasted for 2-4 h then injected with 0.5 U/kg insulin. Blood glucose was measured before injection and every 30 min thereafter, up to 2 h.

### Glycogen Extraction

Small pieces of livers were weighted at dissection. Samples were boiled for 30 min in 500 μl of 30% KOH solution with Vortex every 10 min. 100 μl of 1 M Na₂SO₄ were added when the samples were cooled down. 1.2 ml of 100% ethanol was added, and the samples cooked for 5 min at 95 °C. The samples were then centrifuged for 5 min at 16,000×g. Pellets were washed twice by resuspension in 500 μl of double-distilled water, 1 ml 100% ethanol, and centrifugation. Following the last wash, pellets were air-dried and resuspended in 100 μl of 50 mM sodium acetate at pH 4.8 containing 0.3 mg/ml amyloglucosidase. The samples were incubated at 37 °C overnight on a shaker to facilitate digestion. Fujifilm Autokit glucose assay (997-03001) was used to determine the amount of glycogen by comparing it to a standard curve. The content of glycogen was normalized to tissue weight.

### Glycogen Synthase Activity

Freeze-dried liver tissue (20 g-30 mg) was homogenized in ice-cold buffer (50 mM Tris-HCl (pH 7.8), 100 mM NaF, 10 mM EDTA). Homogenates were centrifuged for 5 min at 3600 g at 4 °C and GYS activity measured in supernatants with 0 or 12 mM of added G6-P (40). Next, the Transcreener®UDP² FI Assay Kit (BellBrook, 3019-A) was used to measure GYS activity by measuring the release of UDP from UDP-glucose.

Measuring Liver Protein Synthesis with O-Propargyl-Puromycin

8-wo Fbp1^{F/F} and Fbp1^{ΔHep} mice were starved for 4-6 h starting at 6 μm and then i.p. injected with 100 μl of a 20 mM solution of O-propargyl-puromycin (OP-Puro, MedChemExpress, HY-15680) in PBS (41). Livers were harvested after 1 h, fixed in formalin and paraffin embedded samples were cut into 5 μm sections. OPP incorporation was detected by staining with Click-&-Go Plus 488 OPP Protein Synthesis Assay Kit (Click Chemistry, #1493), following manufacturer's instructions. Briefly, liver sections were deparaffinized with xylene, hydrated with ethanol and incubated with a reaction cocktail containing copper catalyst, AZDye Azide plus solution and reducing agent for 20 min, while protected from light. DNA was stained with Hoechst 33342 and the sections imaged on a confocal microscope. The intensity of fluorescent-labeled protein-incorporated OPP was quantified by automated analysis of microscopic images with Image J software (NIH).

### Generation and Infection by AAV8-FBP1, AAV8-FBP1^{E98A} and AAV8-Ctrl Virus

pAAV[Exp]-CAG>mFbpIWT:T2A:EGFP:WPRE and pAAV[Exp]CAG>HA/mFbp1^{E98A}:T2A: EGFP:WPRE were constructed by Vectorbuilder. AAV8-mFBP1, AAV8-mFBP1^{E98A} and AAV8-Ctrl viruses were generated by Vectorbuilder. Briefly, 293T cells were co-transfected with indicated vectors and Rep-cap plasmid and helper plasmids. After 50-60 h, cell lysates were harvested and concentrated by PEG precipitation and CsCl gradient ultracentrifugation. 6-8 wo mice were infected with AAV8-mFBP1, mFBP1^{E98A} and AAV8-Ctrl (10¹² virus copies per mouse) via tail vein injection. Liver and serum were collected 3 weeks later.

### Primary Mouse Hepatocytes Isolation

8-wo male Fbp1^{F/F} and Fbp1^{ΔHep} mice were fasted for 14-16 h and primary hepatocytes were isolated using a two-step collagenase perfusion as described (42). Briefly, mice livers were perfused with perfusion buffer (HBSS (Thermo Fisher, 14175095) with 0.5 mM EDTA and 25 mM HEPES) and digested with digestion buffer (HBSS with Ca²⁺ Mg²⁺ (Thermo Fisher, 24020117), 25 mM HEPES and 1 mg/ml Liberase). The hepatocytes were spun down at 50 g for 3 min at 4 °C then purified on a Percoll gradient. The hepatocytes were counted and directly collected or plated on collagen-coated plates for 6 h in plating medium (DMEM low glucose, 5% FBS and 1% Penicillin-streptomycin (PS)). After cell attachment, the plating medium was replaced with maintenance medium (Willams E medium, 2 mM glutamine and 1% PS) for further use.

### Proximity Ligation Assays (PLA)

FBP1:ALDOB:PP2A-C:AKT interactions in liver tissue were detected with an in siWu PLA kit (Duolink® In Situ Red Starter Kit Mouse/Rabbit, Sigma, DUO92101) (43). Briefly, paraffin embedded tissue sections were deparaffinized with 3× washes with xylene, rehydrated with ethanol and subjected to antigen unmasking with citrate buffer at 95-98 °C for 15 min. Tissues were incubated with 3% H₂O₂ for 10 min and then blocked with Duolink blocking solution at 37 °C for 60 min. The samples were incubated with primary antibodies overnight, and then incubated with the PLA Probe, ligase and polymerase following manufacturer's instructions. Tissues were mounted with an in situ mounting medium with DAPI and images captured on a TCS SPE Leica confocal microscope. The results were quantified by counting dots per field in 3 randomly selected fields of view.

### Immunoprecipitation and Mass Spectrometry Analysis

For immunoprecipitation (IP) experiments, cells and livers were harvested in lysis buffer (20 mM Tris-HCl, pH 7.5, 1% NP-40, 137 mM NaCl, 1 mM MgCl₂, 1 mM CaCl₂, 10% glycerol) supplemented with complete protease inhibitor cocktail. Cell and liver lysates were pre-cleared with 30 μl protein G beads (Life Technologies), and then incubated with 2 μg isotype matched IgG control or indicated antibodies on a rocking platform overnight at 4 °C. 50 μl protein G were added and incubated for another 2-3 h. The immunocomplexes were washed 5× with lysis buffer, separated by SDS-PAGE and analyzed by immunoblotting as above.

For mass spectrometry analysis, Fbp1^{ΔHep} mouse livers transduced with AAV-FBP1 or AAV-Control were homogenized in NP-40 lysis buffer. Lysates were IP'ed with HA antibody using magnetic beads. The IP's were washed twice with 1×PBS (Gibco) and resuspended in 150 uL 5% formic acid to denature proteins and remove them from the beads. The supernatants were placed in new tubes and 1.5 mL HPLC-grade water was added and then dried down in a concentrator (Thermo SpeedVac). Dried samples were resuspended in 250 uL 1M urea, reduced with 5 uL of 500 mM dithiothreitol (DTT) and placed on a 47 °C block for 30 minutes. Samples were then cooled to room temperature and 15 μL 500 mM iodoacetamide to alkylate proteins were added and the samples were kept in the dark for 45 minutes. The reactions were quenched with 5 uL 500 mM DTT. 1M Tris-HCl was added to adjust the pH and 5 μL trypsin (2.5 ug, Promega Sequence Grade) was added and the samples digested overnight at 37 °C 80 μL of 1% TFA was added to stop the digestion and the samples were dried down. The samples were then resuspended in 100 μL 0.1% formic acid (FA) desalted using C18 discs in a pipette tip (“Stage Tips”). The eluted peptides were then quantified (Thermo PepQuant) and 9 μg of peptides were transferred to a new tube and dried down. These samples were then resuspended in 9 μL 5% acetonitrile and 5% FA and were placed in MS inserts for proteomic characterization. Sample spectra were collected on a Thermo Fusion mass spectrometer that collected MS data in positive ion mode within the 400 to 1,500 m/z range. Mass spectra raw files were first searched using Proteome Discoverer 2.2 using the built-in SEQUEST search algorithm. Built-in TMT batch correction was enabled for all samples.

### In Vitro Binding Assay

In vitro binding assay was performed as described previously (23). In brief, recombinant GST-PP2A-C protein were incubated with His-AKT, His-ALDOB and His-FBP1 in binding buffer (25 mM Tris-HCl, 200 Mm NaCl, 1 mM EDTA, 0.5% NP-40, 10 μg/μl BSA and 1 mM DTT). GST, AKT or FBP1 antibodies and protein A/G beads were mixed and incubated overnight. The lysates were washed with ice-cold binding buffer for 3 times and boiled before subjected to IB analysis.

### PP2A Dephosphorylation Assay

The PP2A dephosphorylation assay were performed as described (23). Briefly, Huh7 cells stably expressing HA-AKT1 or HA-c-Myc were serum starved for 6 h and stimulated with insulin (100 nM) or EGF (100 ng/ml) for 1 h, respectively. Cells were harvested and AKT1 or c-Myc was IP'ed with an HA antibody. The immunoprecipitates were washed and resuspended in PP2A phosphatase assay buffer (20 mM HEPES, 100 mM NaCl and 3 mM DTT). Together with recombinant ALDOB, FBP1 or FBP1^{E98A} and active PP2A, the AKT1 or c-Myc IPs were incubated at 30 °C for the indicated times and AKT1 and c-Myc phosphorylation was assessed by IB analysis.

### Peptide Synthesis and Treatment

Cell permeable Scrambled (Ctrl) and FBP1 peptides were synthesized by Biomatik. Male BL6 mice were HFD fed for 14 weeks from week 7 postnatally. Mice were i.p. injected with the Ctrl and FBP1 peptides (10 mg/kg each) every 2 days for 2 weeks. Mice were fasted overnight before being subjected to GTT and PTT and fasted 2 h before ITT.

### Protein Structure Predictions and Peptide Docking

AKT1, PP2A-C, ALDOB, FBP1 and FBP1 E7 peptide structure modeling were performed with the ColabFold implantation of AlphaFold (44). The best positions for peptide and protein interactions were explored, and hydrogen bonds and amino acids were identified and labeled in PyMOL (https://pymol.org/2/).

### RNA Isolation and Quantitative Real-Time PCR (Q-PCR)

Total liver RNA was extracted with RNeasy Plus Mini kit (Qiagen #74134) and cDNA was synthesized with SuperScript™ VILO™ cDNA Synthesis Kit (Thermo Fisher Scientific, 11754050) (42). mRNA expression was determined by CFX96 thermal cycler (Biorad). Data are presented as arbitrary units and calculated by comparative CT method (2Ct^{(18r tRNA-gene of interest)}). Primers are listed in Table 3.

### Quantification and Statistical Analysis

Data are presented as mean±SD or mean±SEM as indicated. Differences between mean values were analyzed by unpaired two-tailed Student's t test with GraphPad Prism software. P value<0.05 was considered as significant (*p<0.05, **p<0.01, ***p<0.001, ****p<0.0001, n.s., p≥0.05).

TABLE 1
Primers for constructs used in this study, Related to STAR Methods.

| Insert | Forward Primer (5′-3′) | Reverse Primer (5′-3′) |
|---|---|---|
| FBP1-R158W | GCAACCAGGCTGGAACCTGG | AGAGCATCCTTCTCAGAAGGC |
| | T | |
| FBP1-G164S | GGTGGCAGCCAGCTACGCACT | AGGTTCCGGCCTGGTTGC |
| FBPI-N213K | ACAGCCTTAAGGAGGGCTACG | AGATTTTACCTTTCTTTTTTATCT |
| | | TCACATC |
| FBP1-Q229 | TGAGTACATCTAGAGGAAGAA | GTGACGGCAGGGTCAAAG |
| | GTTC | |
| FBP1-G294E | AAGGCTGGGGAAATGGCCAC | CTCCATGACGTAGGCCATG |
| | C | |
| FBP1-G320R | GGTGATCTTGAGATCCCCCGA | GGCGCCCTCTGGTGAATG |
| | C | |
| FBP1-L329P | CTCGAGTTCCCGAAGGTGTAT | CACGTCGTCGGGGGATCC |
| | GAGAAGC | |
| FBP1-C39dup | CACTGCACAGCAGTCAAAGC | CAGGAGCGAGTTGAGCAGCTGG |
| | C | |

TABLE 2
shRNA and sgRNA sequences used in this study,
Related to STAR Methods.

| shRNA and sgRNA | Target Sequences (5′-3′) |
|---|---|
| sgALDOB#1 | CACCGGATCACACCCCCGATGCTC |
| sgALDOB#2 | CACCGGATTTCTCGGAACTGCCGG |
| shPP2A-C#1 | TGGAACTTGACGATACTCTAA |
| shPP2A-C#2 | CCCATGTTGTTCTTTGTTATT |
| shFBP1#1 | CGACCTGGTTATGAACATGTT |
| shFBP1#2 | CAGCAGTCAAAGCCATCTCTT |

TABLE 3
Quantitative PCR primers used in this study, Related to STAR Methods.

| Gene | Forward Primer (5′-3′) | Reverse Primer (5′-3′) |
|---|---|---|
| Acly | GCCAGCGGGAGCACATC | CTTTGCAGGTGCCACTTCATC |
| Acaca | TGACAGACTGATCGCAGAGAAAG | TGGAGAGCCCCACACACA |
| Fasn | GCTGCGGAAACTTCAGGAAAT | AGAGACGTGTCACTCCTGGACTT |
| Hmgcs1 | GCCGTGAACTGGGTCGAA | GCATATATAGCAATGTCTCCTGCA |
| | | A |
| Hmgcr | CTTGTGGAATGCCTTGTGATTG | AGCCGAAGCAGCACATGAT |
| Tkt | CGAAACCCTCACAATGATCG | TTCCTCAGGTTCAGCAGCTC |
| Pgd | ATGCCAGGAGGGAACAAAG | GTTCTCCGGTTCCCACTTTT |
| Glut2 | TGCTGGCCTCAGCTTTATTC | TTTCTTTGCCCTGACTTCCTC |
| Pkm2 | GCTA TTCGAGGAACTCCGCC | AAGGTACAGGCACTACACGC |
| Me1 | GATGATAAGGTCTTCCTCACC | TTACTGGTTGACTTTGGTCTGT |
| Mthfd2 | ACAGATGGAGCTCACGAACG | TGCCAGCGGCAGATATTACA |
| Ppat | AATAGCTGTGGCCCATAACG | ACGTGGAAAGCCCAATACC |
| Gbe1 | GCGATCATGGAACATGCTTACTAT | CCATAACGACTTGAAGCTGCAA |
| Cpt1α | TGTCCAAGTATCTGGCAGTCG | CATAGCCGTCATCAGCAACC |
| Cpt2 | ATCGTACCCACCATGCACTAC | CTGTCATTCAAGAGAGGCTTCTG |
| Srebp1c | GGAGCCATGGATTGCACATT | GGCCCGGGAAGTCACTGT |
| Cd36 | TCCTCTGACATTTGCAGGTCTATC | AAAGGCATTGGCTGGAAGAA |

### Results

### Hypoglycemia, Hepatomegaly, Fatty Liver, and Hyperlipidemia in Fasted Fbp1^{ΔHep} Mice

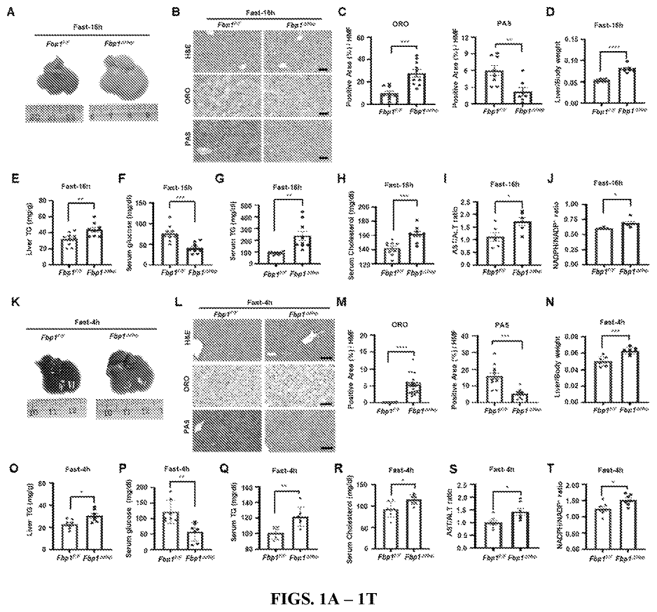

To understand the metabolic impact of FBP1 deficiency and its biochemical sequalae, Applicant crossed Fbp1^{F/F} and Alb-Cre mice to generate Fbp1^{ΔHep} mice. At 8 weeks-of-age and after a 16 h overnight fast, Fbp1^{ΔHeP}, but not Fbp1^{F/F}, mice exhibited hepatomegaly, hepatosteatosis, severe hypoglycemia, hyperlipidemia, and signs of liver damage (elevated AST:ALT ratio) (FIGS. 1A-1I). Weights of other tissues were not altered (FIG. 8B). Hepatomegaly was due to hypertrophy rather than hyperplasia (FIG. 8C and FIG. 8D). Elevated lipolysis was unlikely to be the cause of hepatosteatosis because adipose tissue mRNAs encoding hormone sensitive lipase (HSL) and adipose triglyceride lipase (ATGL) were insignificantly affected by Fbp1 ablation (FIG. 8E). Despite hypoglycemia, the Fbp1^{ΔHep} livers contained more NADPH, ATP, and acetyl-CoA, which support lipid synthesis, than Fbp1^{F/F} livers (FIG. 1J, FIGS. 8F-8H). No genotype-related differences in serum insulin or glucagon were observed in the fast state, although serum lactate was elevated in fasted Fbp1^{ΔHep} mice (FIGS. 81-8K). Consistent with Periodic Acid Schiff (PAS) staining, hepatic glycogen, and G6-P were reduced (FIG. 1B, FIG. 8L, and FIG. 8M). Interestingly, even a mere 4 h fast caused mild hepatomegaly, hepatosteatosis, hypoglycemia, hyperlipidemia, signs of liver injury and elevated hepatic NADPH:NADP ratio and ATP in Fbp1^{ΔHep} mice (FIGS. 1K-1T, FIG. 8N, and FIG. 8O). No significant differences in weights of other tissues or lipolytic enzyme mRNAs were observed (FIG. 8P and FIG. 8Q). Immunoblot (IB) analysis showed insignificant differences in HSL and ATGL phosphorylation in epididymal white adipose tissue (eWAT) after the 4 h fast (FIG. 8R), further ruling out enhanced lipolysis as the basis for the observed metabolic abnormalities. The short 4 h fast still led to elevated serum lactate in Fbp1^{ΔHep} mice but little genotype-related differences in serum insulin and glucagon (FIG. 8S-8U). Lastly, hepatic glycogen and G6-P were significantly lower in Fbp1^{ΔHep} mice than in Fbp1^{F/F} mice (FIG. 1L, FIG. 8V, and FIG. 8W). In short, the metabolic phenotypes of hepatic steatosis, hypoglycemia, hepatomegaly, and hyperlipidemia associated with FBP1 deficiency appeared quickly within 4 h of fasting.

### Fasted Fbp1^{ΔHep} Mice Exhibit Enhanced AKT-mTOR Signaling and De Novo Lipogenesis

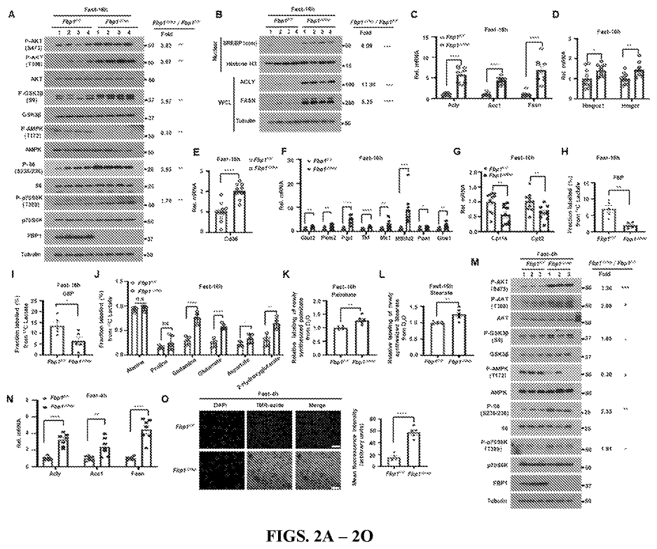

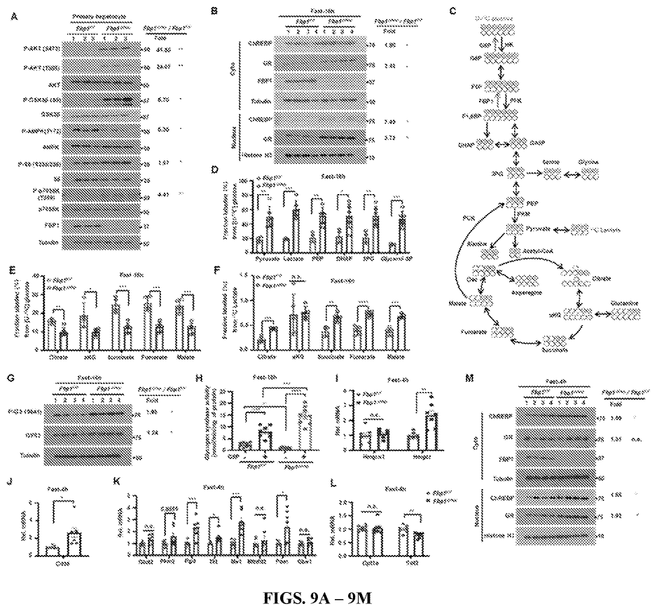

Fasted (16 h) Fbp1^{ΔHep} livers and primary mouse hepatocytes showed enhanced AKT S473 and T308 and GSK3β S9 phosphorylations, loss of AMPK T172 phosphorylation and elevated S6 and p70 S6 kinase (p70S6K) phosphorylations, suggesting mTORC1 activation (FIG. 2A and FIG. 9A), Consistent with hepatosteatosis, previous findings (11), and mTORC1 and p70S6K activation (14), nuclear/mature (m) SREBP1c was strongly elevated in fasted Fbp1^{ΔHep} livers, along with its targets, ATP citrate lyase (ACLY), acetyl CoA carboxylase 1 (ACC1) and fatty acid synthase (FASN) and the SREBP2 targets HMG CoA synthase 1 (HMGCS1) and reductase (HMGCR) (FIGS. 2B-2D). The lipid transporter CD36, Glut2, pyruvate kinase M2 (PKM2), phosphogluconate dehydrogenase (PGD), transketolase (TKT), malic enzyme 1 (ME1), methylenetetrahydrofolate dehydrogenase 2 (MTHFD2), phosphoribosyl pyrophosphate amidotransferase (PPAT) and 1,4-α-glucan branching enzyme 1 (GBE1) mRNAs were elevated, whereas carnitine palmitoyl transferases 1a and 2 (CPT1a, CPT2) mRNAs, involved in β-oxidation, were reduced (FIGS. 2E-2G). Carbohydrate response element binding protein (ChREBP), whose overexpression promotes hepatic steatosis while improving insulin sensitivity (15), was also elevated in the cytoplasm and nucleus of Fbp1^{ΔHep} livers and so was glucocorticoid receptor (GR) (FIG. 9B).

GC-MS based metabolomic analysis of ¹³C-glucose loaded livers revealed elevated glycolytic flux and decreased TCA cycle activity in fasted Fbp1^{ΔHep} livers (FIGS. 9C-9E), explaining the increase in serum lactate. ¹³C-lactate tracing showed reduced lactate flux to F6-P and G6-P but enhanced lactate flux into TCA cycle intermediates and amino acids (FIGS. 2H-2J, FIG. 9C, and FIG. 9F), correlating with reduced liver glycogen. D₂O tracing confirmed elevated de novo lipogenesis (DNL) in fasted Fbp1^{ΔHep} mice (FIG. 2K and FIG. 2L). Curiously, glycogen synthase 2 (GYS2) and its phosphorylated form (P-GS) were elevated in fasted Fbp1^{ΔHep} livers (FIG. 9G) despite their low glycogen content and the increase in inhibitory GSK3 phosphorylation, which should have increased GYS2 activity (16). Without being bound by theory, this suggested that diminished Fbp1^{ΔHep} liver glycogen stores could be due to low G6-P, the key allosteric activator of GYS2 (17), which is generated by PGI from F6-P, whose production from lactate via GNG was reduced. Indeed, GYS activity was significantly lower in Fbp1^{ΔHep} liver lysates relative to Fbp1^{F/F} liver lysates (FIG. 9H). This difference was indeed due to lower G6-P in the Fbp1^{ΔHep} liver, because when exogenous G6-P was added to the reaction mix GYS activity was higher in Fbp1^{ΔHep} liver lysates, consistent with higher GYS2 abundance.

After a 4 h fast, Fbp1^{ΔHep} livers also showed elevated AKT activation, GSK3β S9 phosphorylation, S6 and p70S6K phosphorylation and reduced AMPK activation (FIG. 2M). SREBP1 target mRNAs encoding ACLY, ACC1 and FASN were moderately elevated, along with the SREBP2 target HMGCR, the lipid transporter CD36, and the metabolic enzymes PKM2, PGD, TKT, ME1 and PPAT, whereas CPT2 mRNA was decreased (FIG. 2N and FIGS. 9I-9L). Consistent with mTORC1 activation and hepatomegaly, protein synthesis measured with O-propargyl-puromycin (OP-puro) was strongly elevated in 4 h-fasted Fbp1^{ΔHep} hepatocytes (FIG. 2O) and so were nuclear ChREBP and GR (FIG. 9M). Thus, the FBP1 deficiency results in rapid fasting-induced upregulation of AKT-mTOR signaling, lipogenesis and protein synthesis.

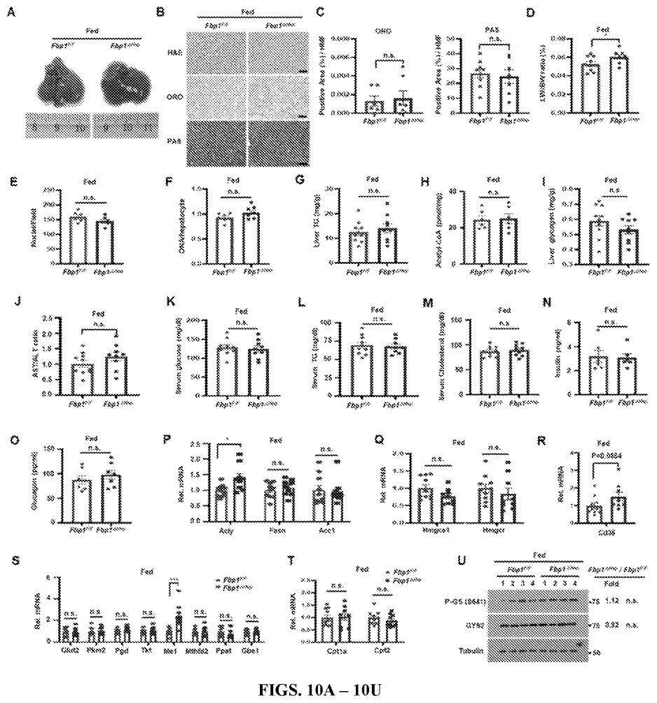

Of note, in the fed state Fbp1^{ΔHep} livers were barely different from Fbp1^{F/F} livers, although the Fbp1^{ΔHep} liver exhibited slight hepatomegaly, somewhat lower glycogen and marginally elevated liver damage (FIGS. 10A-10J). Serum parameters also did not differ between the two genotypes (FIGS. 10K-10O). Fbp1^{ΔHep} livers also showed increased ACLY and ME1 mRNAs, but mRNAs encoding other metabolic genes did not differ from those in Fbp1^{F/F} livers (FIGS. 10P-10T). No changes in GYS2 and P-GS amounts were observed (FIG. 10U).

AKTi and mTORCi Attenuate Lipogenesis and Protein Synthesis but not Hypoglycemia

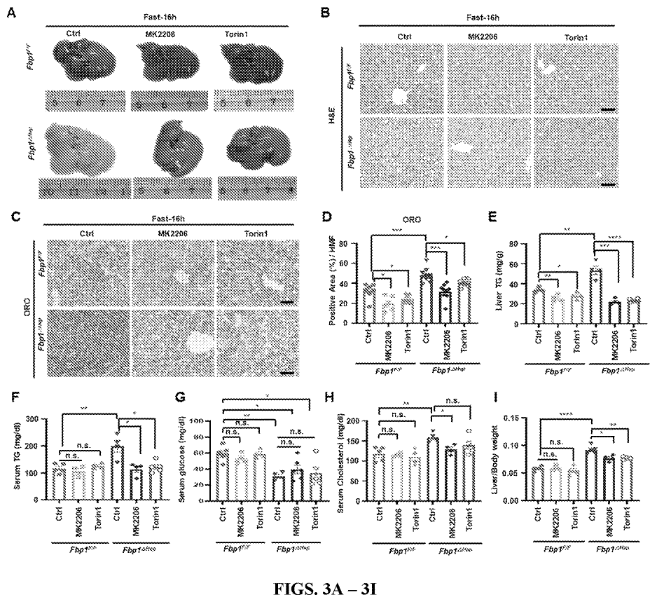

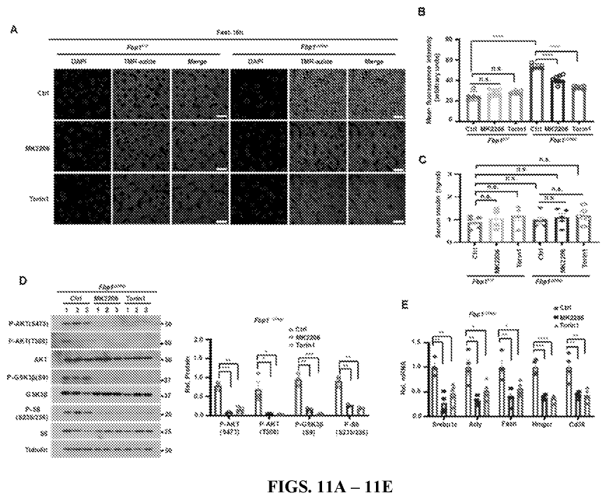

To determine whether elevated AKT or mTORC1 activities contribute to the metabolic abnormalities in fasted Fbp1^{ΔHep} mice, Applicant treated the mice with AKT (MK2206) or mTORC1/2 (Torin1) inhibitors 28 and 4 h before fasting. Both inhibitors prevented hepatosteatosis and hypertriglyceridemia, reducing them to levels seen in drug-treated fasted Fbp1^{F}F mice, but had no effect on hypoglycemia (FIGS. 3A-3G). Serum cholesterol was reduced by MK2206 but not by Torin1 (FIG. 3H). The two inhibitors partially abrogated hepatomegaly (FIGS. 3A and 3I) and blunted the fasting-induced increase in protein synthesis but did not alter serum insulin (FIGS. 11A-11C). As expected, MK2206 and Torin1 (which also inhibits mTORC2 that acts upstream to AKT) blocked fasting induced AKT, ribosomal protein S6 and GSK3β phosphorylation and blunted SREBP1c, ACLY, FASN, HMGCR and CD36 mRNA expression (FIGS. 11D and 11E).

### Catalytically Inactive FBP1 Prevents Hepatomegaly, Steatosis, and Hyperlipidemia

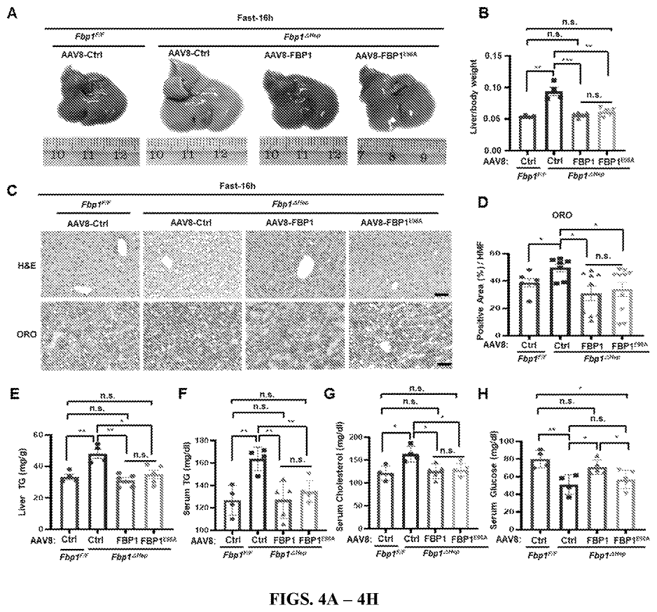

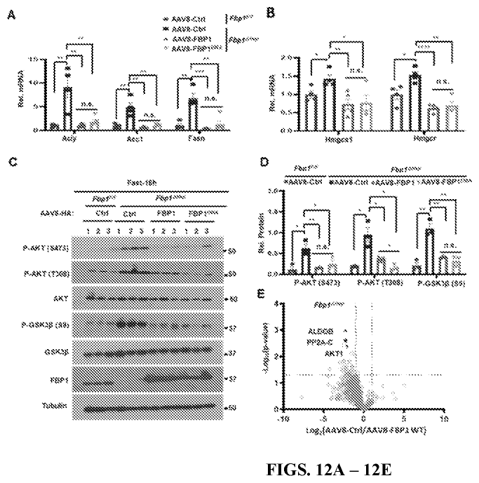

In addition to catalyzing GNG, FBP1 non-enzymatically inhibits HIF-1 activity (6). AAV8-mediated re-expression of WT FBP1 or catalytically inactive FBP1^{E98A} in Fbp1^{ΔHep} hepatocytes had an effect akin to AKTi treatment, blunting fasting-induced hepatomegaly, hepatosteatosis and hyperlipidemia and reducing lipogenic gene expression, although as expected FBP1^{E98A} did not obliterate hypoglycemia (FIGS. 4A-4H, FIG. 12A and FIG. 12B). AAV8 mediated FBP1 or FBP1^{E98A} re-expression largely suppressed AKT activation and GSK3β S9 phosphorylation (FIG. 12C and FIG. 12D).

To elucidate how FBP1 regulated AKT activation, Applicant used mass spectrometry (MS) as an unbiased tool to identify FBP1 binding proteins. ALDOB, AKT1 and PP2A-C ranked as the top FBP1 interacting partners present in HA-FBP1 immunoprecipitates (IPs) isolated from AAV8-FBP1 transduced Fbp1^{ΔHeP} livers (FIG. 12E). Fbp1^{ΔHep} livers transduced with AAV8-Ctrl served as a negative control. Of note, no PP2A regulatory subunits co-IP'ed with FBP1.

FBP1 Associates with PP2A-C and ALDOB to Bind AKT and Inhibit its Activation

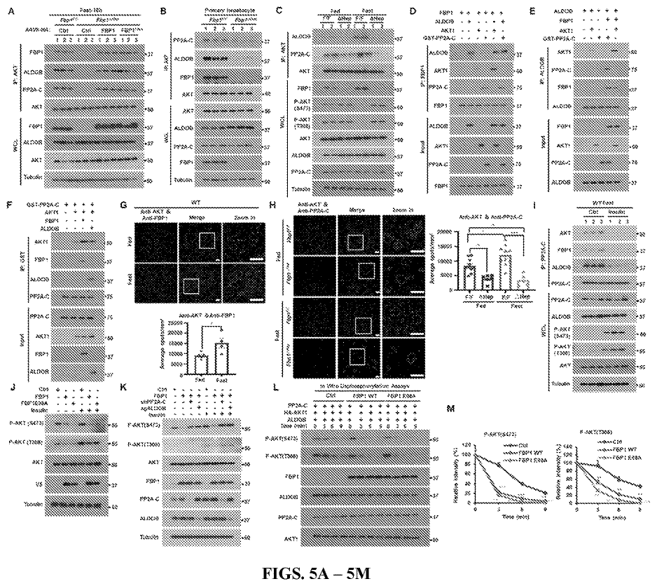

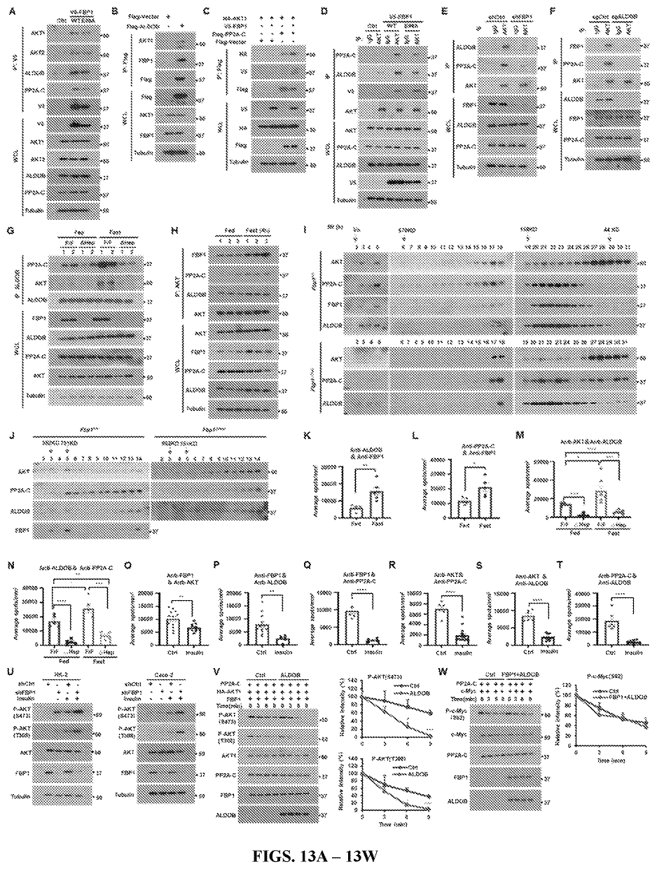

Next, Applicant examined whether PP2A-C and ALDOB were part of the AKT inhibitory mechanism triggered by FBP1. FBP1-deficient individuals exhibit mild hereditary fructose intolerance (HFI) (8, 10), a metabolic disorder associated with ALDOB deficiency (OMIM; 229600), which blocks F1,6-P2 breakdown (8, 19). ALDOB deficiency also causes hepatomegaly, hepatosteatosis and liver damage (20, 21) This phenotypic overlap suggested that FBP1 may regulate AKT activity through its interaction with ALDOB, which was also detected in previous studies (22). Consistent with our MS analysis, ALDOB was reported to recruit PP2A-C to AKT in HCC cells and enhance AKT dephosphorylation (23). Indeed, Applicant confirmed that ectopically expressed WT and catalytically inactive FBP1^{E98A} interacted with AKT1, AKT2, ALDOB and PP2A-C in 293T cells, and that ectopic ALDOB IPs contained endogenous AKT1 and FBP1 (FIG. 13A and FIG. 13B). Likewise, ectopic PP2A-C interacted with AKT1 and FBP1, which enhanced the AKT1-PP2A-C interaction (FIG. 13C). Ectopic WT or FBP1^{E98A} expression in Huh7 cells potentiated the interaction between AKT, ALDOB and PP2A-C, but FBP1 or ALDOB silencing disrupted the interactions between AKT, PP2A-C, ALDOB and FBP1 (FIGS. 13D-13F). These interactions were also confirmed in mouse liver; AKT IPs from Fbp1^{F/F} or Fbp1^{ΔHep} livers reconstituted with FBP1 or FBP1^{E98A} contained FBP1, ALDOB and PP2A-C (FIG. 5A). Similar results were obtained when AKT was IP'ed from primary mouse hepatocytes, in which the absence of FBP1 diminished the association of AKT with ALDOB and PP2A-C (FIG. 5B), indicating that FBP1 plays a pivotal role in complex formation. Formation of the FBP1-dependent FBP1:PP2A-C:ALDOB:AKT complex was enhanced by fasting (FIG. 5C, FIG. 13G, and FIG. 13H). In vitro binding experiments confirmed assembly of the FBP1:PP2A-C:ALDOB:AKT complex using purified recombinant proteins, revealing that FBP1 first binds ALDOB and PP2A-C, and weakly to AKT1 (FIGS. 5D-5F). Addition of ALDOB and PP2A-C enhanced AKT1 binding and although ALDOB and PP2A-C interacted with AKT1 these interactions were enhanced by FBPL Gel filtration chromatography of Fbp1^{F/F} liver lysates revealed co-elution of FBP1, AKT, PP2A-C and ALDOB in a high molecular weight fraction, averaging 750-800 kDa, which was not seen in Fbp1^{ΔHep} lysates (FIG. 13I and FIG. 13J).

Proximity ligation assays (PLA) confirmed formation of the FBP1:PP2A-C:ALDOB:AKT complex in mouse liver and the pivotal role of FBP1 in coordinating ALDOB and PP2A-C recruitment to AKT (FIG. 5G,FIG. 5H, and FIG. 13K-FIG. 13N). The interactions between FBP1, ALDOB, PP2A-C and AKT were enhanced by fasting and weakened by feeding or insulin (FIGS. 5G-5I and FIGS. 13K-13T). FBP1 knockdown in the kidney epithelial cell line HK-2 or the colon carcinoma-derived CaCo-2 cell line also augmented AKT activation with and without insulin (FIG. 13U). Ectopic expression of either WT FBP1 or FBP1^{E98A} in HCC cells inhibited basal and insulin-stimulated AKT S473 phosphorylation in a PP2A-C- and ALDOB-dependent manner (FIG. 5J and FIG. 5K). In vitro dephosphorylation assay using active AKT1 as a substrate showed that either FBP1 variant stimulated time-dependent S473 and T308 dephosphorylation in an ALDOB dependent manner (FIG. 5L, FIG. 5M, and FIG. 13V). Dephosphorylation of another PP2A substrate was not affected by FBP1 (FIG. 13W).

### Fbp1^{ΔHep} Mice are Hyperresponsive to Insulin

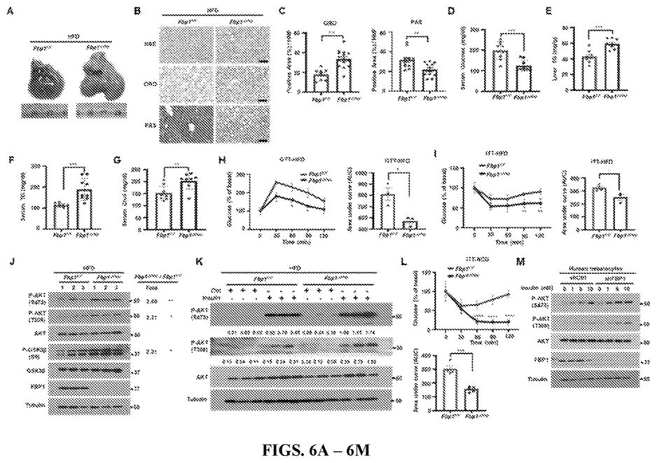

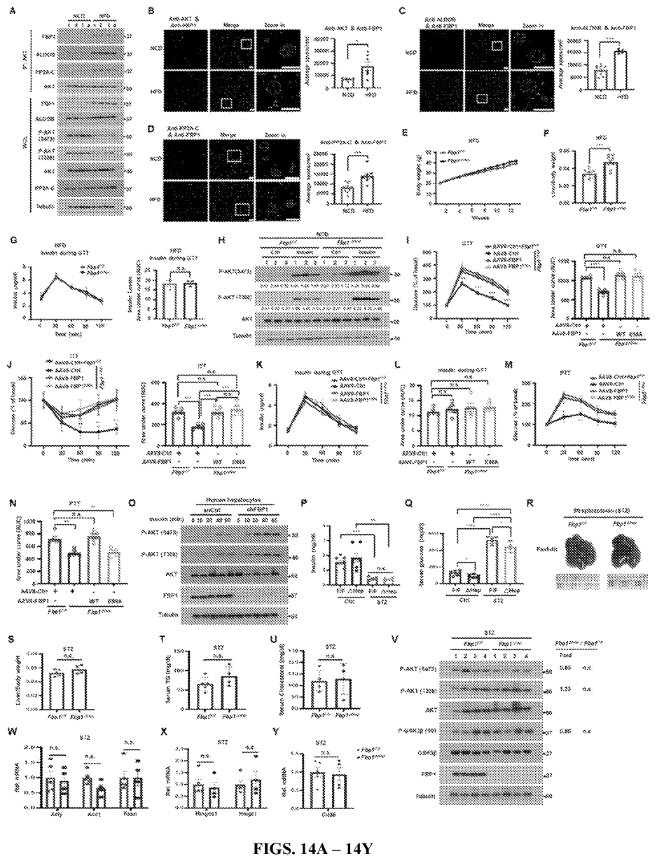

As people and mice with obesity show insulin resistance which interferes with AKT activation, Applicant checked how HFD consumption affects the FBP1-PP2A-C-ALDOB-AKT complex. WB and PLA showed that HFD enhanced FBP1 and ALDOB expression as well as complex formation and inhibited AKT activation in WT mice (FIGS. 14A-14D). Applicant also fed Fbp1^{F/F} and Fbp1^{ΔHep} mice HFD for 12 weeks. While weight gain was nearly identical between the two, Fbp1^{ΔHep} mice exhibited higher liver to body weight ratio (FIG. 14E and FIG. 14F), hepatomegaly, enhanced hepatosteatosis, but less liver glycogen, than Fbp1^{F/F} mice (FIGS. 6A-6C). Although HFD-fed Fbp1^{ΔHep} mice were protected from obesity-related hyperglycemia, their liver and serum triglycerides (TG) and cholesterol were elevated (FIGS. 6D-6G). Glucose tolerance test (GTT) showed that HFD-fed Fbp1^{ΔHep} mice were more glucose tolerant than Fbp1^{F/F} mice, although insulin levels did not differ between the two (FIG. 6H and FIG. 14G). Insulin tolerance test (ITT) confirmed insulin hypersensitivity in HFD-fed Fbp1^{ΔHep} mice (FIG. 6I), whose liver contained more phosphorylated AKT and GSK3β than the Fbp1^{F/F} liver (FIG. 6J). Insulin administration to either HFD- or normal chow (NCD)-fed Fbp1^{F/F} and Fbp1^{ΔHep} mice resulted in higher AKT phosphorylation, under both HFD and NCD feeding and a considerable and sustained drop in blood glucose in NCD-fed Fbp1^{ΔHep} mice (FIG. 6K, FIG. 6L, and FIG. 14H). GTT and ITT indicated that both FBP1 and FBP1^{E98A} re-expression decreased glucose tolerance and insulin hypersensitivity (FIG. 14I and FIG. 14J), even though insulin levels during GTT did not differ between the two (FIG. 14K and FIG. 14L). Fbp1^{ΔHep} mice were less pyruvate tolerant and this was reversed by AAV-FBP1, but not AAV-FBP1^{E98A}, re-expression (FIG. 14M and FIG. 14N). Suppression of insulin-induced AKT activation was also seen in human hepatocytes, where FBP1 silencing enhanced insulin induced AKT activation in a dose- and time-dependent manner (FIG. 6M and FIG. 14O).

Insulin concentrations are higher in the fed state relative to the fasted state, making us wonder how fasting triggers AKT activation in Fbp1^{ΔHep} mice. Postulating that the fasting-induced increase in AKT activation, steatosis and hepatomegaly in Fbp1^{ΔHep} mice could be due to FA-stimulated insulin secretion as a consequence of fasting-induced lipolysis (24), Applicant injected Fbp1^{F/F} and Fbp1^{ΔHep} mice with high dose streptozotocin (STZ) to destroy insulin producing β cells (25). As expected, STZ treatment reduced blood insulin and increased blood glucose, which was slightly lower in Fbp1^{ΔHep} mice than in Fbp1^{F/F} mice after a 4 h fast (FIG. 14P and FIG. 14Q). Importantly, STZ treatment blocked fasting-induced hepatomegaly, hyperlipidemia, AKT and GSK3β phosphorylation, and the increase in ACLY, ACC1, FASN, HMGCS1, HMGCR and CD36 mRNA expression (FIGS. 14R-14Y), indicating that the liver phenotypes manifested by fasted Fbp1^{ΔHep} mice depend on insulin secretion from β cells.

### A Complex Disrupting FBP1-Derived Peptide Ameliorates Insulin Resistance

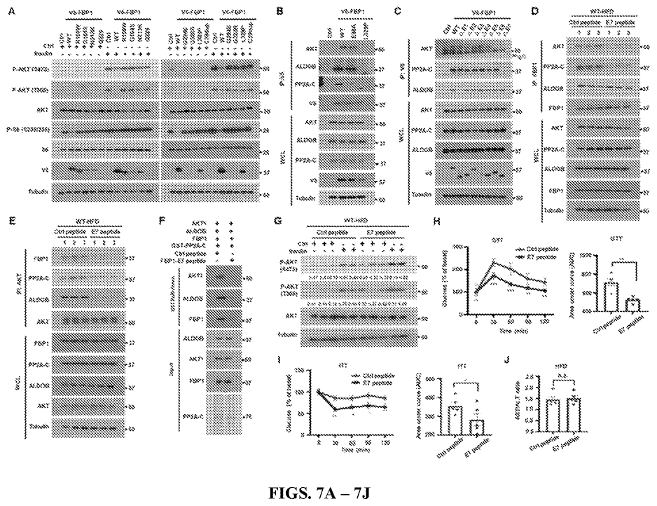

In addition to deletion and nonsense mutations that block FBP1 expression, human FBP1 deficiency can be caused by missense mutations (9, 26). Applicant stably expressed FBP1 missense mutants in Huh7 cells and found most of them to be poorly expressed and devoid of catalytic activity (FIG. 7A and FIG. 15A). Those variants that were expressed, R158W, G164S, N213K and L329P, no longer inhibited insulin stimulated AKT phosphorylation (FIG. 7A). Particularly interesting was FBP1^{L329P} in exon 7, which was relatively well expressed with only a partial loss of FBP1 catalytic activity but completely devoid of AKT inhibitory activity (FIG. 7A, FIG. 15A, and FIG. 15B). Unlike WT FBP1 and FBP1^{E98A}, FBP1^{L329P} did not associate with AKT, ALDOB and PP2A-C (FIG. 7B). To further delineate FBP1 sequences mediating these interactions Applicant used FBP1 deletion mutants, ΔE1-ΔE7 (6), each lacking one FBP1 exon (E) (FIG. 15B). ΔE6 and ΔE7 did not bind AKT nor inhibited its activity and ΔE7 did not interact with PP2A-C, but ΔE1 was only defective in ALDOB binding (FIG. 7C and FIG. 15C). These results suggest that FBP1 assembles the AKT inhibitory complex through separate and distinct contacts with AKT, PP2A-C and ALDOB. To test whether a synthetic E7 peptide that binds AKT, or PP2A-C can disrupt the FBP1:PP2A-C:ALDOB:AKT complex and lead to AKT activation, Applicant synthesized a peptide encompassing FBP1 AA 275 to 300 preceded by a TAT-derived cell penetrating peptide (FIG. 15D). According to AlphaFold structural prediction and docking analysis, the E7 peptide is alpha helical, assuming the same fold it has within native FBP1, and is capable of differential association with the N-termini of AKT1 and PP2A-C but not with ALDOB (FIGS. 15E-15G).

As FBP1 mRNA is highly expressed in patients with obesity and T2D (GSE15653 and GSE64998) (FIGS. 15H-15J), Applicant examined whether FBP1:PP2A-C:ALDOB:AKT complex disruption reverses insulin resistance. Applicant injected the E7 peptide or a scrambled peptide to HFD-fed BL6 mice. Strikingly, the C-terminal peptide disrupted the association of FBP1 with AKT and PP2A-C, and AKT with FBP1, PP2A-C and ALDOB (FIG. 7D and FIG. 7E). GST pulldown and PLA confirmed that the FBP1 E7 peptide reduced the association of PP2A-C with FBP1, AKT1 and ALDOB, as well as the AKT-PP2A-C, AKT-FBP1 and FBP1-PP2A-C interactions, but had no effect on the FBP1-ALDOB interaction (FIG. 7F and FIGS. 15K-15N), which is mediated by FBP1 E1. The E7 peptide enhanced basal and insulin-stimulated AKT activation in HFD-fed WT mice (FIG. 7G). In vitro dephosphorylation assays showed that the E7 peptide retarded AKT dephosphorylation by PP2A-C (FIG. 15O). Importantly, E7 peptide treatment restored glucose tolerance and insulin sensitivity to HFD-fed mice, modestly reducing gluconeogenesis, lowering their blood glucose, and increasing liver glycogen and lipids with no effect on serum TG, weight gain or FBP1 catalytic activity (FIGS. 7H-7J and FIGS. 15P-15W). These results establish FBP1 as an important attenuator of insulin signaling in WT mice, suggesting that this non-enzymatic FBP1 function can be exploited in the development of insulin sensitizers for treatment of individuals with obesity-related insulin resistance.

### DISCUSSION

Until now, the sole metabolic function assigned to FBP1 is the conversion of F1,6-P₂ to F6-P, a critical reaction in GNG (7, 27). In cancer cell nuclei, however, FBP1 was found to inhibit HIF1_{α} activity through protein-protein interactions (6). Here Applicant show that cytoplasmic FBP1 has another critical and physiologically relevant metabolic function that does not depend on its enzymatic activity, formation of a regulatory complex containing PP2A-C and ALDOB that binds AKT and blunts its activation. Discovery of this heretofore unrecognized function, in which FBP1 serves as a “safety valve” that prevents excessive insulin signaling and balances glucose and lipid metabolism, was enabled by generation of an accurate mouse model of human FBP1 deficiency (OMIM; 229700). Like FBP1 deficient infants, Fbp1^{ΔHep} mice, which lack hepatic FBP1 since an early age, are generally normal but exhibit rapid and severe hypoglycemia, hepatomegaly, hepatosteatosis, hyperlipidemia and liver damage upon fasting. Hypoglycemia and low liver glycogen stores are due to lack of FBP1 catalytic activity, which is needed for GNG and generation of G6-P, the allosteric activator of glycogen synthase. By contrast, the different liver pathologies and hyperlipidemia depend on insulin stimulated AKT hyperactivation and not on FBP1 catalytic activity. Accordingly, these pathologies, but not hypoglycemia, are prevented by re-expression of catalytically inactive FBP1, inhibition of AKT and to a lesser extent by inhibition of mTORC1. In addition to stimulation of protein synthesis (28), a likely driver of hepatomegaly, mTORC1 activates the lipogenic transcription factor SREBP1c (14), accounting for increased lipogenesis and hyperlipidemia. Enhanced AKT activation may also contribute to some of the long-term metabolic abnormalities seen in older FBP1 deficient individuals (8). A somewhat different phenotype that includes liver damage, fibrosis, and enhanced hepatocyte tumorigenesis, but little or no effect on glucose tolerance, was observed after AAV-Cre mediated FBP1 ablation in older (24-wo) Fbp1^{F/F} mice (11). These phenotypic differences are most likely due to the timing of FBP1 ablation and the evaluation of glucose tolerance in lean vs. HFD-fed mice.

While causing AKT hyperactivation in fasted and insulin-treated mice, hepatocyte FBP1 deficiency protects HFD-fed mice from glucose intolerance. The catalytically inactive FBP1^{E98A} variant binds ALDOB and PP2A-C and inhibits AKT activation as effectively as the native protein, but human FBP1 missense mutants that are not severely destabilized, L329P, R158W, G164S and N213K, are devoid of AKT inhibitory activity. One of the residues affected by these mutations, L329, is not a part of the catalytic pocket (29, 30) and is associated with only a modest decrease in FBPase activity, underscoring the disconnect between loss of GNG and insulin hyperresponsiveness. Mass spectrometry, IP, gel filtration and PLA demonstrated that FBP1 is the lynchpin responsible for formation of the FBP1:PP2A-C:ALDOB:AKT regulatory complex. While insulin destabilizes this complex to facilitate AKT activation, HFD stimulates FBP1 expression and complex formation, suggesting that elevated FBP1 expression is a previously unknown contributor to HFD-induced insulin resistance. Although more precise understanding of the interactions between FBP1, AKT, PP2A-C and ALDOB requires detailed structural analysis, our biochemical analysis suggests that the regulatory function of FBP1 depends on the recruitment of PP2A-C to AKT, rather than other phosphoproteins. Of note, a synthetic peptide derived from FBP1 E7 can be docked onto AKT and PP2A-C and disrupt their interactions with native FBP1. By potentiating AKT activation, this peptide serves as an insulin mimic, capable of reversing obesity-induced glucose intolerance, and further demonstrating the physiological relevance of the FBP1 nucleated AKT inhibitory complex. Although like insulin secretagogues and sensitizers E7 treatment modestly potentiates hepatosteatosis, the restoration of insulin sensitivity outweighs the increased risk of hepatosteatosis, which could be circumvented by co-treatment with β-oxidation inducing uncouplers (31). The non-enzymatic regulatory function of FBP1 probably appeared early in vertebrate evolution, as it modulates liver energy storage and lipid production that are highly important in egg laying animals. In addition to causing potentially fatal insulin hyperresponsiveness, the loss of FBP1 regulatory function may also increase the risk of non-alcoholic steatohepatitis.

### EQUIVALENTS

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

### REFERENCES

1. Saltiel, A. R. (2021). Insulin signaling in health and disease. The Journal of clinical investigation 131.
2. Petersen, M. C., Vatner, D. F., and Shulman, G. I. (2017). Regulation of hepatic glucose metabolism in health and disease. Nat. Rev. Endocrinol 13, 572-587.
3. Hatting, M., Tavares, C. D. J., Sharabi, K., Rines, A. K., and Puigserver, P. (2018). Insulin regulation of gluconeogenesis. Ann. N. Y. Acad. Sci. 1411, 21-35.
4. Santoleri, D., and Titchenell, P. M. (2019). Resolving the Paradox of Hepatic Insulin Resistance. Cell Mol. Gastroenterol. Hepatol. 7, 447-456.
5. Hay, N. (2011). Interplay between FOXO, TOR, and Akt. Biochim. Biophys. Acta 1813, 1965-1970.
6. Li, B., Qiu, B., Lee, D. S., Walton, Z. E., Ochocki, J. D., Mathew, L. K., Mancuso, A., Gade, T. P., Keith, B., Nissim, I., et al. (2014). Fructose-1,6-bisphosphatase opposes renal carcinoma progression. Nature 513, 251-255.
7. Hunter, R. W., Hughey, C. C., Lantier, L., Sundelin, E. I., Peggie, M., Zeqiraj, E., Sicheri, F., Jessen, N., Wasserman, D. H., and Sakamoto, K. (2018). Metformin reduces liver glucose production by inhibition of fructose-1-6-bisphosphatase. Nat. Med. 24, 1395-1406.
8. Gorce, M., Lebigot, E., Arion, A., Brassier, A., Cano, A., De Lonlay, P., Feillet, F., Gay, C., Labarthe, F., Nassogne, M. C., et al. (2022). Fructose-1,6-bisphosphatase deficiency causes fatty liver disease and requires long-term hepatic follow-up. J. Inherit. Metab. Dis. 45, 215-222.
9. Salih, R. M., Mohammed, E. A., Alhashem, A. M., Mohamed, S., and Al-Aqeel, A. I. (2020). Fructose-1,6-bisphosphatase deficiency with confirmed molecular diagnosis. An important cause of hypoglycemia in children. Saudi. Med. J. 41, 199-202.
10. Moey, L. H., Abdul Azize, N. A., Yakob, Y., Leong, H. Y., Keng, W. T., Chen, B. C., and Ngu, L. H. (2018). Fructose-1,6-bisphosphatase deficiency as a cause of recurrent hypoglycemia and metabolic acidosis: Clinical and molecular findings in Malaysian patients. Pediatr. Neonatol. 59, 397-403.
11. Li, F., Huangyang, P., Burrows, M., Guo, K., Riscal, R., Godfrey, J., Lee, K. E., Lin, N., Lee, P., Blair, I. A., et al. (2020). FBP1 loss disrupts liver metabolism and promotes tumorigenesis through a hepatic stellate cell senescence secretome. Nat. Cell Biol. 22, 728-739.
12. Schultze, S. M., Hemmings, B. A., Niessen, M., and Tschopp, O. (2012). PI3K/AKT, MAPK and AMPK signalling. protein kinases in glucose homeostasis. Expert Rev. Mol. Med. 14, el.
13. Grill, V., and Qvigstad, E. (2000). Fatty acids and insulin secretion. Br. J. Nutr. 83 Suppl 1, S79-84.
14. Bakan, I., and Laplante, M. (2012). Connecting mTORC1 signaling to SREBP-1 activation. Curr. Opin. Lipidol. 23, 226-234.
15. Benhamed, F., Denechaud, P. D., Lemoine, M., Robichon, C., Moldes, M., Bertrand-Michel, J., Ratziu, V., Serfaty, L., Housset, C., Capeau, J., et al. (2012). The lipogenic transcription factor ChREBP dissociates hepatic steatosis from insulin resistance in mice and humans. J. Clin. Invest. 122, 2176-2194.
16. Beurel, E., Grieco, S. F., and Jope, R. S. (2015). Glycogen synthase kinase-3 (GSK3): regulation, actions, and diseases. Pharmacol. Ther. 148, 114-131.
17. Villar-Palasí, C., and Guinovart, J. J. (1997). The role of glucose 6-phosphate in the control of glycogen synthase. Faseb. j. 11, 544-558.
18. Herman, M. A., and Birnbaum, M. J. (2021). Molecular aspects of fructose metabolism and metabolic disease. Cell Metab. 33, 2329-2354.
19. Simons, N., Debray, F. G., Schaper, N.C., Kooi, M. E., Feskens, E. J. M., Hollak, C. E. M., Lindeboom, L., Koek, G. H., Bons, J. A. P., Lefeber, D. J., et al. (2019). Patients With Aldolase B Deficiency Are Characterized by Increased Intrahepatic Triglyceride Content. J. Clin. Endocrinol. Metab. 104, 5056-5064.
20. Oppelt, S. A., Sennott, E. M., and Tolan, D. R. (2015). Aldolase-B knockout in mice phenocopies hereditary fructose intolerance in humans. Mol. Genet. Metab. 114, 445-450.
21. Di Dato, F., Spadarella, S., Puoti, M. G., Caprio, M. G., Pagliardini, S., Zuppaldi, C., Vallone, G., Fecarotta, S., Esposito, G., Iorio, R., et al. (2019). Daily Fructose Traces Intake and Liver Injury in Children with Hereditary Fructose Intolerance. Nutrients 11.
22. Droppelmann, C. A., Sáez, D. E., Asenjo, J. L., Yáñez, A. J., García-Rocha, M., Concha, II, Grez, M., Guinovart, J. J., and Slebe, J. C. (2015). A new level of regulation in gluconeogenesis: metabolic state modulates the intracellular localization of aldolase B and its interaction with liver fructose-1,6-bisphosphatase. Biochem. J. 472, 225-237.
23. He, X., Li, M., Yu, H., Liu, G., Wang, N., Yin, C., Tu, Q., Narla, G., Tao, Y., Cheng, S., et al. (2020). Loss of hepatic aldolase B activates Akt and promotes hepatocellular carcinogenesis by destabilizing the Aldob/Akt/PP2A protein complex. PLoS Biol. 18, e3000803.
24. Crespin, S. R., Greenough, W. B., 3rd, and Steinberg, D. (1969). Stimulation of insulin secretion by infusion of free fatty acids. J. Clin. Invest. 48, 1934-1943.
25. Graham, M. L., Janecek, J. L., Kittredge, J. A., Hering, B. J., and Schuurman, H. J. (2011). The streptozotocin-induced diabetic nude mouse model: differences between animals from different sources. Comp. Med. 61, 356-360.
26. Emecen Sanli, M., Cengiz, B., Kilic, A., Ozsaydi, E., Inci, A., Okur, I., Tumer, L., Lebigot, E., and Ezgu, F. (2022). Fructose 1,6 bisphosphatase deficiency: outcomes of patients in a single center in Turkey and identification of novel splice site and indel mutations in FBP1. J. Pediatr. Endocrinol. Metab.
27. Leithner, K. (2020). Epigenetic Marks Repressing Gluconeogenesis in Liver and Kidney Cancer. Cancer research 80, 657-658.
28. Bhaskar, P. T., and Hay, N. (2007). The two TORCs and Akt. Dev. Cell. 12, 487-502.
29. Ruf, A., Tetaz, T., Schott, B., Joseph, C., and Rudolph, M. G. (2016). Quadruple space-group ambiguity owing to rotational and translational noncrystallographic symmetry in human liver fructose-1,6-bisphosphatase. Acta Crystallogr. D. Struct. Biol. 72, 1212-1224.
30. Huang, Y., Xu, Y., Song, R., Ni, S., Liu, J., Xu, Y., Ren, Y., Rao, L., Wang, Y., Wei, L., et al. (2020). Identification of the New Covalent Allosteric Binding Site of Fructose-1,6-bisphosphatase with Disulfiram Derivatives toward Glucose Reduction. J. Med. Chem. 63, 6238-6247.
31. Goedeke, L., Peng, L., Montalvo-Romeral, V., Butrico, G. M., Dufour, S., Zhang, X. M., Perry, R. J., Cline, G. W., Kievit, P., Chng, K., et al. (2019). Controlled-release mitochondrial protonophore (CRMP) reverses dyslipidemia and hepatic steatosis in dysmetabolic nonhuman primates. Sci. Transl. Med. 11.
32. Kim, J. Y., Garcia-Carbonell, R., Yamachika, S., Zhao, P., Dhar, D., Loomba, R., Kaufman, R. J., Saltiel, A. R., and Karin, M. (2018). ER Stress Drives Lipogenesis and Steatohepatitis via Caspase-2 Activation of SiP. Cell 175, 133-145.e115.
33. Todoric, J., Di Caro, G., Reibe, S., Henstridge, D. C., Green, C. R., Vrbanac, A., Ceteci, F., Conche, C., McNulty, R., Shalapour, S., et al. (2020). Fructose stimulated de novo lipogenesis is promoted by inflammation. Nat. Metab. 2, 1034-1045.
34. Gu, L., Zhu, Y., Lin, X., Lu, B., Zhou, X., Zhou, F., Zhao, Q., Prochownik, E. V., and Li, Y. (2021). The IKKβ-USP30-ACLY Axis Controls Lipogenesis and Tumorigenesis. Hepatology 73, 160-174.
35. Zwingmann, C., and Bilodeau, M. (2006). Metabolic insights into the hepatoprotective role of N-acetylcysteine in mouse liver. Hepatology 43, 454-463.
36. Sengupta, A., Ghosh, S., Sharma, S., and Sonawat, H. M. (2020). Early Perturbations in Glucose Utilization in Malaria-Infected Murine Erythrocytes, Liver and Brain Observed by Metabolomics. Metabolites 10.
37. Gray, L. R., Sultana, M. R., Rauckhorst, A. J., Oonthonpan, L., Tompkins, S. C., Sharma, A., Fu, X., Miao, R., Pewa, A. D., Brown, K. S., et al. (2015). Hepatic Mitochondrial Pyruvate Carrier 1 Is Required for Efficient Regulation of Gluconeogenesis and Whole-Body Glucose Homeostasis. Cell Metab. 22, 669-681.
38. Huh, J. Y., Reilly, S. M., Abu-Odeh, M., Murphy, A. N., Mahata, S. K., Zhang, J., Cho, Y., Seo, J. B., Hung, C. W., Green, C. R., et al. (2020). TANK-Binding Kinase I Regulates the Localization of Acyl-CoA Synthetase ACSL1 to Control Hepatic Fatty Acid Oxidation. Cell Metab. 32, 1012-1027.e1017.
39. Scott, D. A. (2021). Analysis of Melanoma Cell Glutamine Metabolism by Stable Isotope Tracing and Gas Chromatography-Mass Spectrometry. Methods Mol. Biol. 2265, 91-110.
40. Bouskila, M., Hunter, R. W., Ibrahim, A. F., Delattre, L., Peggie, M., van Diepen, J. A., Voshol, P. J., Jensen, J., and Sakamoto, K. (2010). Allosteric regulation of glycogen synthase controls glycogen synthesis in muscle. Cell Metab. 12, 456-466.
41. Liu, J., Xu, Y., Stoleru, D., and Salic, A. (2012). Imaging protein synthesis in cells and tissues with an alkyne analog of puromycin. Proc. Natl. Acad. Sci. USA 109, 413-418.
42. He, F., Antonucci, L., Yamachika, S., Zhang, Z., Taniguchi, K., Umemura, A., Hatzivassiliou, G., Roose-Girma, M., Reina-Campos, M., Duran, A., et al. (2020). NRF2 activates growth factor genes and downstream AKT signaling to induce mouse and human hepatomegaly. J. Hepatol. 72, 1182-1195.
43. Su, H., Yang, F., Fu, R., Li, X., French, R., Mose, E., Pu, X., Trinh, B., Kumar, A., Liu, J., et al. (2021). Cancer cells escape autophagy inhibition via NRF2-induced macropinocytosis. Cancer Cell 39, 678-693.e611.
44. Jumper, J., Evans, R., Pritzel, A., Green, T., Figurnov, M., Ronneberger, O., Tunyasuvunakool, K., Bates, R., Židek, A., Potapenko, A., et al. (2021). Highly accurate protein structure prediction with AlphaFold. Nature 596, 583-589.

1. An isolated recombinant peptide comprising one or more of an FBP1 E1-E7 fragment or an equivalent thereof and a cell penetrating peptide.

2. The isolated recombinant peptide of claim 1, wherein the FBP1 E1-E7 fragment comprises the FBP1 E7 or an equivalent thereof.

3. The isolated recombinant peptide of claim 2, wherein the FBP1 E1-E7 fragment comprises XGKLRLLYECNPMAYVMEKAGGMATTG, wherein X is no amino acid or an optional linker peptide (SEQ ID NO: 2) or wherein the FBP1 E1-E7 fragment consists of XGKLRLLYECNPMAYVMEKAGGMATTG. wherein X is no amino acid or an optional linker petide (SEQ ID NO: 2).

4. (canceled)

5. The isolated recombinant peptide of claim 2, wherein the FBP1 E7 peptide comprises GKLRLLYECNPMAYVMEKAGGMATTG (SEQ ID NO: 1), or an equivalent thereof, or wherein the FBP1 E7 peptide consists of GKLRLLYECNPMAYVMEKAGGMATTG (SEQ ID NO: 1), or an equivalent thereof.

6. (canceled)

7. The recombinant peptide of claim 1, wherein the cell penetrating peptide comprises the peptide TAT (SEQ ID NO: 6) or YGRKKRRQRRR (SEQ ID NO: 4).

8. The recombinant peptide of claim 1, comprising TATXGKLRLLYECNPMAYVMEKAGGMATTG, wherein X is any amino acid or an optional linker peptide (SEQ ID NO: 2).

9. The recombinant peptide of claim 1, comprising GKLRLLYECNPMAYVMEKAGGMATTGXTAT, wherein X is no amino acid or an optional linker peptide (SEQ ID NO: 3).

10. The recombinant peptide of claim 1, comprising YGRKKRRQRRRGGGKLRLLYECNPMAYVMEKAGGMATTG (SEQ ID NO: 5), or an equivalent thereof.

11. The recombinant peptide of claim 1, consisting of YGRKKRRQRRRGGGKLRLLYECNPMAYVMEKAGGMATTG (SEQ ID NO: 5), or an equivalent thereof.

12. The recombinant peptide of claim 1, further comprising a detectable label.

13. A polynucleotide encoding the peptide of claim 1.

14. The polynucleotide of claim 13, wherein the polynucleotide is DNA or RNA.

15. A vector comprising the polynucleotide of claim 14, optionally operably linked to regulatory elements, optionally wherein the vector is a plasmid or a viral vector.

16. (canceled)

17. An isolated host cell comprising one or more of the recombinant peptide of claim 1.

18. A method to disrupt an interaction of AKT and/or PP2A-C and disrupt their interactions with FBP1, the method comprising contacting a composition comprising AKT and/or PP2A, and FBP1 with an effective amount of the peptide of claim 1.

19. (canceled)

20. A method for one or more of: activating AKT, sensitizing insulin, or reversing obesity-induced glucose intolerance in a subject in need thereof, the method comprising administering to the subject an effective amount of the recombinant peptide of claim 1.

21-22. (canceled)

23. A composition comprising a carrier and one or more of the recombinant peptide of claim 1.

24. The composition of claim 23, wherein the carrier is a pharmaceutically acceptable carrier.
